(19) United States
(12) Patent Application Publication
(10) Pub. No.: US 2012/0101271 A1
(43) Pub. Date: Apr. 26, 2012

(54) DIAMINE SYNTHESIS VIA CATALYTIC C-H AMINATION OF AZIDES

(76) Inventors: X. Peter Zhang, Tampa, FL (US); Hongjian Lu, Tampa, FL (US)

(21) Appl. No.: 13/278,932

(22) Filed: Oct. 21, 2011

Related U.S. Application Data
(60) Provisional application No. 61/405,899, filed on Oct. 22, 2010.

(51) Int. Cl. C07D 285/16; C07C 209/62; C07D 417/04; C07C 381/00; C07D 207/27

(52) U.S. Cl. 544/6; 552/5; 548/551; 544/8; 564/367

(57) ABSTRACT

Selective intramolecular C—H amination via metalloradical activation of azides: synthesis of 1,3-diamines under neutral and nonoxidative conditions. One aspect of the present invention is the synthesis of 1,3-diamines by intramolecular C—H amination of sulfamoyl azides. More specifically, sulfamoyl azides may be selectively aminated via metalloradical activation of azides, preferably with Co(II) porphyrins. In a particularly preferred embodiment, the Co(II) porphyrin is a D₂ₕ-symmetric porphyrin.

### CROSS-REFERENCE TO RELATED APPLICATIONS

The present application claims the benefit of U.S. Provisional Application Ser. No. 61/405,899, filed Oct. 22, 2010, which is hereby incorporated by reference herein in its entirety.

### STATEMENT OF GOVERNMENT RIGHTS

This invention was made with Government support under grant number NSF #0711024, awarded by the National Science Foundation, Division of Chemistry. The Government has certain rights in the invention.

### BACKGROUND OF THE INVENTION

Amino groups exist ubiquitously in natural products and synthetic molecules, playing key roles in a wide range of important applications. Consequently, immense efforts have been devoted to develop efficient and selective processes for the preparation of amines.¹ Among different approaches, catalytic amination of abundant C—H bonds, based on metal-mediated nitrene insertion pathway, represents one of the most general and direct methods for installing nitrogen functionalities.² The promise of this approach as a synthetically useful methodology has recently been demonstrated with a number of intramolecular C—H amination processes through the combined use of Rh(II)₂-based catalysts and iminoiodane nitrene sources.^{2,3} Notably, Du Bois and coworkers have elegantly demonstrated that N-Boc-sulfamides could be selectively converted to cyclic sulfonamides by [Rh₂(esp)₂] in combination with PhI(OAc)₂ and MgO, providing access to synthetically useful 1,3-diamines.⁴ The Rh(II)₂-based intramolecular amination was shown to be effective for both secondary and tertiary C—H bonds with high stereospecificity and diastereoselectivity. However, amination of strong primary C—H bonds had yet to be demonstrated.⁵ Moreover, the catalytic system was unsuitable for simple N-alkyl-sulfamides as they were oxidatively degraded by the stoichiometric oxidant, PhI(OAc)₂.⁴

### SUMMARY OF THE INVENTION

As stable metalloradicals, cobalt(II) complexes of porphyrins [Co(Por)] have emerged as a new class of catalysts for C—H amination.⁶ The Co(II)-based metalloradical amination (MRAm) is different from the commonly-studied Rh₂-system as it can operate effectively with various azides as substrates without the need of terminal oxidants and other additives.⁷⁻¹¹ To further validate the utility of Co(II)/azide-based C—H amination methodology, we envisioned a general strategy for the synthesis of 1,3-diamines from monoamines through the key step of intramolecular C—H amination of sulfamoyl azides by [Co(Por)] (Scheme 1: Cobalt(II)-Catalyzed Intramolecular C—H Amination of Sulfamoyl Azides under Neutral and Nonoxidative Conditions: General Synthetic Strategy for 1,3 Diamine Derivatives). We report herein a Co(II)-based catalytic system that is highly effective for the intramolecular 1,6-C—H amination of sulfamoyl azides, which furnishes the 6-membered cyclic sulfonamides in excellent yields. In addition to excellent regioselectivity, high diastereoselectivity and stereospecificity can be achieved with the catalytic system. Together with N₂ as the only byproduct, the Co(II)-catalyzed amination is highlighted by its operational simplicity as it can proceed under neutral and nonoxidative conditions without the need of other reagents. Consequently, it enjoys a high degree of functional group tolerance and can be applied to substrates with varied substituents, such as oxidizable amide and sulfide groups. Besides secondary and tertiary C—H bonds, the current catalytic system features with effective amination of strong primary C—H bonds.

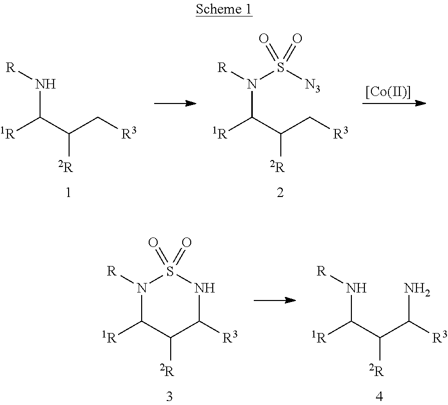

Among the various aspects of the present invention, therefore, may be noted the provision of a sulfamoyl azide corresponding to Formula SA-1:

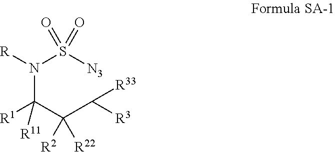

wherein

R is hydrogen, hydrocarbyl, substituted hydrocarbyl, heterocyclo, or, in combination with R¹ or R¹¹ and carbon atoms to which they are attached, form a heterocyclo,

R¹ is hydrogen, hydrocarbyl, substituted hydrocarbyl, heterocyclo, or in combination with R and carbon atoms to which R and R¹, respectively, are attached, form a heterocyclo,

R² is hydrogen, hydrocarbyl, substituted hydrocarbyl, heterocyclo, or in combination with R¹ is or R¹¹ and the carbon atoms to which R² and R¹¹, respectively, are attached, form a carbocyclo or a heterocyclo,

R³ is hydrogen, hydrocarbyl, substituted hydrocarbyl, or heterocyclo,

R¹¹ is hydrogen, hydrocarbyl, substituted hydrocarbyl, heterocyclo, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,

R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and

R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

Another aspect of the present invention is the provision of a cyclic sulfonamide corresponding to Formula S-1:

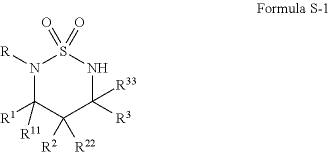

wherein

R is hydrogen, hydrocarbyl, substituted hydrocarbyl, heterocyclo, or, in combination with R¹ or R¹¹ and carbon atom to which they are attached, form a heterocyclo,

R¹ is hydrogen, hydrocarbyl, substituted hydrocarbyl, heterocyclo, or in combination with R and carbon atoms to which R and R¹, respectively, are attached, form a heterocyclo,

R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,

R³ is hydrogen, hydrocarbyl, substituted hydrocarbyl, or heterocyclo,

R¹¹ is hydrogen, hydrocarbyl, substituted hydrocarbyl, heterocyclo, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,

R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and

R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

Another aspect of the present invention is the provision of a diamine corresponding to Formula A-1:

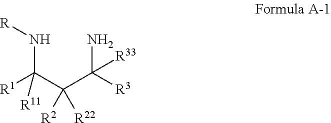

wherein

R is hydrogen, hydrocarbyl, substituted hydrocarbyl, heterocyclo, or, in combination with R¹ or R¹¹ and carbon atom to which they are attached, form a heterocyclo,

R¹ is hydrogen, hydrocarbyl, substituted hydrocarbyl, heterocyclo, hydrocarbyloxy, hydroxy, protected hydroxy, nitro, acyl, or in combination with R and carbon atoms to which R and R¹, respectively, are attached, form a heterocyclo,

R² is hydrogen, hydrocarbyl, substituted hydrocarbyl, heterocyclo, hydrocarbyloxy, hydroxy, protected hydroxy, nitro, acyl, or in combination with R¹¹ and the carbon atoms to which R² and R¹¹, respectively, are attached, form a carbocyclo or a heterocyclo,

R³ is hydrogen, hydrocarbyl, substituted hydrocarbyl, heterocyclo, hydrocarbyloxy, hydroxy, protected hydroxy, nitro, or acyl,

R¹¹ is hydrogen, hydrocarbyl, substituted hydrocarbyl, heterocyclo, hydrocarbyloxy, hydroxy, protected hydroxy, nitro, acyl, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,

R²² is hydrogen, hydrocarbyl, substituted hydrocarbyl, heterocyclo, hydrocarbyloxy, hydroxy, protected hydroxy, nitro, acyl, or in combination with R³³ and the carbon atoms to which R²² and R³³, respectively, are attached, form a carbocyclo or a heterocyclo, and

R³³ is hydrogen, hydrocarbyl, substituted hydrocarbyl, heterocyclo, hydrocarbyloxy, hydroxy, protected hydroxy, nitro, acyl, or in combination with R²² and the carbon atoms to which R²² and R³³, respectively, are attached, form a carbocyclo or a heterocyclo.

Another aspect of the invention is a process for the preparation of a cyclic sulfonamide comprising the intramolecular C—H amination of a sulfamoyl azide in the presence of a cobalt porphyrin catalyst. In a preferred embodiment, the sulfamoyl azide corresponds to Formula SA-1 and the cyclic sulfonamide corresponds to Formula S-1.

Another aspect of the invention is the preparation of a diamine, the process comprising the transamination of a cyclic sulfonamide. In a preferred embodiment, the cyclic sulfonamide corresponds to Formula S-1 and the diamine corresponds to Formula A-1.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

The term “acyl,” as used herein alone or as part of another group, denotes the moiety formed by removal of the hydroxyl group from the group —COOH of an organic carboxylic acid, e.g., RC(O)—, wherein R is R₁, R₁O—, R₁R₂N— or R₁S—, R₁ is hydrocarbyl, heterosubstituted hydrocarbyl, or heterocyclo and R₂ is hydrogen, hydrocarbyl or substituted hydrocarbyl.

The term “acyloxy,” as used herein alone or as part of another group, denotes an acyl group as described above bonded through an oxygen linkage (O), e.g., RC(O)O wherein R is as defined in connection with the term “acyl.”

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “alkoxy” or “alkoxyl” as used herein alone or as part of another group denote any univalent radical, RO— where R is an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The terms “carbocyclo” or “carbocyclic” as used herein alone or as part of another group denote optionally substituted, fully saturated or unsaturated, monocyclic or bicyclic, aromatic or nonaromatic groups having no heteroatom in the ring(s). Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “diazo” or “azo” as used herein alone or as part of another group denote an organic compound with two linked nitrogen compounds. These moieties include without limitation diazomethane, ethyl diazoacetate, and t butyl diazoacetate.

The term “electron acceptor” as used herein denotes a chemical moiety that accepts electrons. Stated differently, an electron acceptor is a chemical moiety that accepts either a fractional electronic charge from an electron donor moiety to form a charge transfer complex, accepts one electron from an electron donor moiety in a reduction-oxidation reaction, or accepts a paired set of electrons from an electron donor moiety to form a covalent bond with the electron donor moiety.

The terms “halogen” or “halo” as used herein alone or as part of another group denote chlorine, bromine, fluorine, and iodine.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The term “heteroatom” as used herein denotes atoms other than carbon and hydrogen.

The terms “heterocyclo” and “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainded of the molecule through a carbon or heteroatom. Exemplary heterocyclo include heteroaromatics as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein alone or as part of another group denote organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, as alkaryl, alkenaryl, and alkynaryl. Unless otherwise indicated, these moieties preferably comprise 1 to 20 carbon atoms.

The term porphyrin refers to a compound comprising a fundamental skeleton of four pyrrole nuclei united through the α-positions by four methane groups to form the following macrocyclic structure:

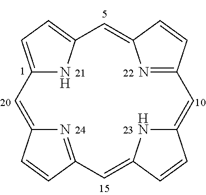

The “substituted hydrocarbyl” moieties described herein, e.g., the substituted alkyl, the substituted alkenyl, the substituted alkynyl, and the substituted aryl moieties, are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substitutents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

### DETAILED DESCRIPTION OF THE PREFERRED EMBODIMENTS

One aspect of the present invention is the synthesis of 1,3-diamines by intramolecular C—H amination of sulfamoyl azides. More specifically, sulfamoyl azides may be selectively aminated via metalloradical activation of azides, preferably with Co(II) porphyrins. In a particularly preferred embodiment, the Co(II) porphyrin is a D₂ₕ-symmetric porphyrin.

In accordance with a preferred embodiment, a sulfamoyl azide is converted to a cyclic sulfonamide as illustrated in Reaction Scheme A:

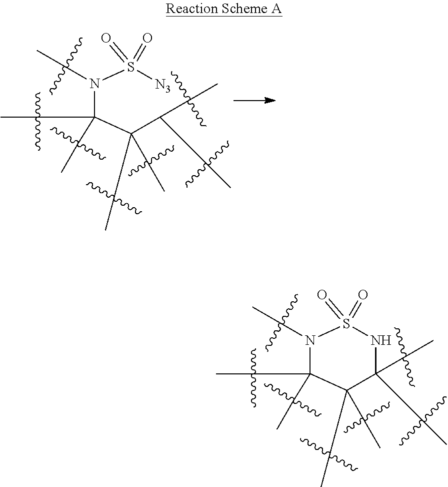

in the presence of a Co(II) porphyrin catalyst, preferably a D₂ₕ-symmetric porphyrin under neutral and non-oxidizing conditions. In one such preferred embodiment, the sulfamoyl azide corresponds to Formula SA-1 and the cyclic sulonamide corresponds to Formula S-1:

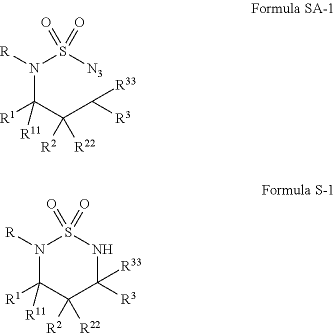

wherein R, R¹, R², R³, R¹¹, R²² and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one embodiment R is alkyl (e.g., methyl, ethyl, propyl, butyl, or pentyl), substituted alkyl (e.g., acyl or trifluoromethyl substituted methyl, ethyl, propyl, butyl or pentyl), aralykl (e.g., C₆H₅CH₂—). By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R¹¹ is hydrogen, hydrocarbyl, substituted hydrocarbyl, heterocyclo, hydrocarbyloxy, hydroxy, protected hydroxy, nitro, or acyl. By way of further example, in one such embodiment R¹¹ is hydrogen, hydrocarbyl, substituted hydrocarbyl, or heterocyclo. By way of further example, in one such embodiment R¹ and R¹¹ are independently hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo. By way of further example, in one such embodiment R³ and R³³ are independently hydrogen, alkyl (e.g., methyl, ethyl, propyl, butyl or pentyl), heterosubstituted alkyl (e.g., heterosubstituted methyl, ethyl, propyl, butyl or pentyl), optionally substituted aryl (e.g., optionally substituted phenyl), or heterocyclo.

In one embodiment, the sulfamoyl azide in Reaction Scheme A corresponds to Formula SA-2 and the cyclic sulonamide in Reaction Scheme A corresponds to Formula S-2:

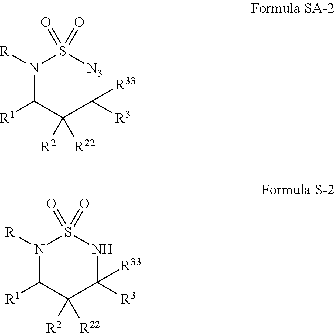

wherein R, R¹, R², R³, R²² and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the sulfamoyl azide in Reaction Scheme A corresponds to Formula SA-2-2 and the cyclic sulonamide in Reaction Scheme A corresponds to Formula S-2-2:

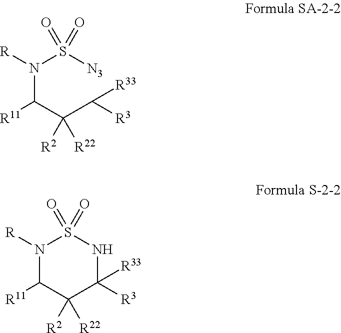

wherein R, R¹, R², R³, R²² and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹¹ is hydrogen, hydrocarbyl, substituted hydrocarbyl, heterocyclo, hydrocarbyloxy, hydroxy, protected hydroxy, nitro, or acyl. By way of further example, in one such embodiment R¹¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the sulfamoyl azide in Reaction Scheme A corresponds to Formula SA-3 and the cyclic sulonamide in Reaction Scheme A corresponds to Formula S-3:

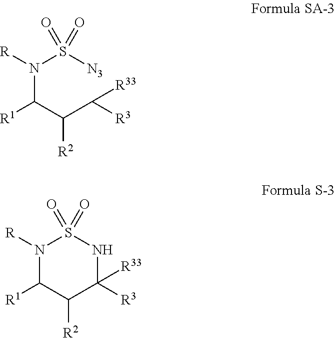

wherein R, R¹, R², R³, and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the sulfamoyl azide in Reaction Scheme A corresponds to Formula SA-4 and the cyclic sulonamide in Reaction Scheme A corresponds to Formula S-4:

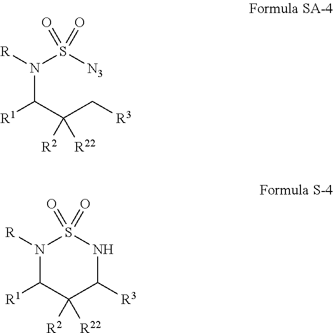

wherein R, R¹, R², R³, and R²² are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In one embodiment, the sulfamoyl azide in Reaction Scheme A corresponds to Formula SA-5 and the cyclic sulonamide in Reaction Scheme A corresponds to Formula S-5:

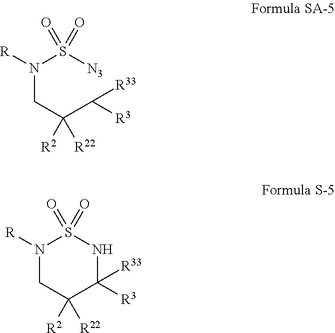

wherein R, R², R³, R²² and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the sulfamoyl azide in Reaction Scheme A corresponds to Formula SA-6 and the cyclic sulonamide in Reaction Scheme A corresponds to Formula S-6:

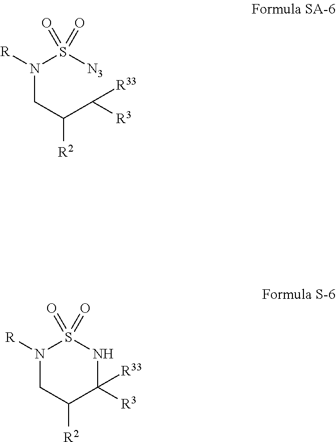

wherein R, R², R³, and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the sulfamoyl azide in Reaction Scheme A corresponds to Formula SA-7 and the cyclic sulonamide in Reaction Scheme A corresponds to Formula S-7:

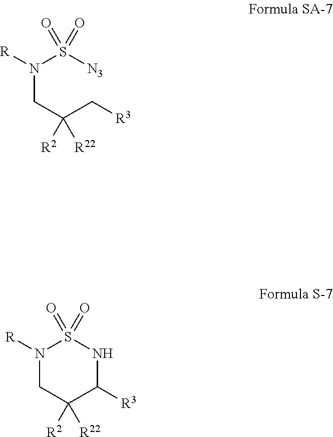

wherein R, R², R³, and R²² are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In one embodiment, the sulfamoyl azide in Reaction Scheme A corresponds to Formula SA-8 and the cyclic sulonamide in Reaction Scheme A corresponds to Formula S-8:

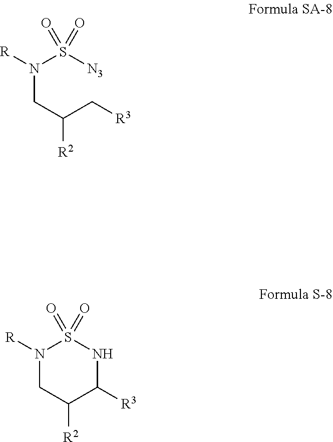

wherein R, R², and R³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In one embodiment, the sulfamoyl azide in Reaction Scheme A corresponds to Formula SA-9 and the cyclic sulonamide in Reaction Scheme A corresponds to Formula S-9:

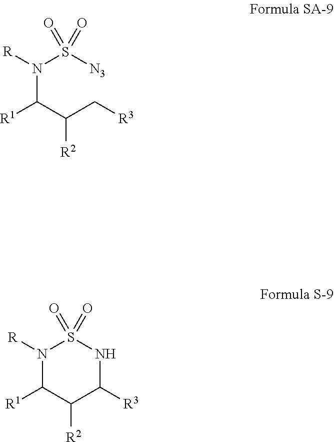

wherein R, R¹, R², and R³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In accordance with a preferred embodiment, a cyclic sulfonamide is converted to a 1,3-diamine as illustrated in Reaction Scheme B:

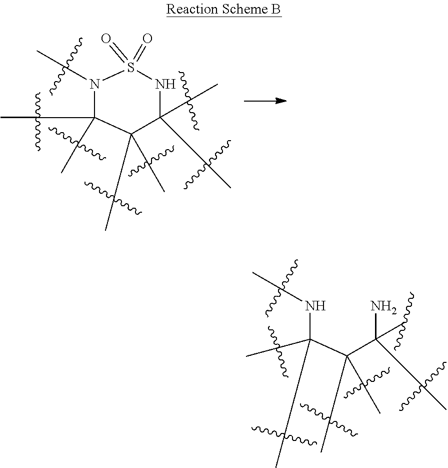

by transamination of the cyclic sulfonamide in the presence of an amine, preferably a primary amine such as diaminopropane under neutral conditions. Alternatively, the cyclic sulfonamide may be transaminated in the presence of an alcohol, such as a diol, under neutral conditions. In one preferred embodiment, the cyclic sulfonamide corresponds to Formula S-1 and the 1,3-diamine corresponds to Formula A-1:

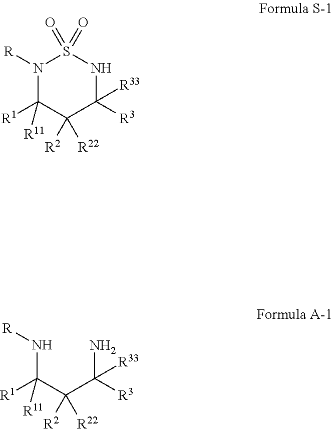

wherein R, R¹, R², R³, R¹¹, R²² and R³³ are as previously defined in connection with Formula S-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R¹¹ is hydrogen, hydrocarbyl, substituted hydrocarbyl, heterocyclo, hydrocarbyloxy, hydroxy, protected hydroxy, nitro, or acyl. By way of further example, in one such embodiment R¹¹ is hydrogen, hydrocarbyl, substituted hydrocarbyl, or heterocyclo. By way of further example, in one such embodiment R¹ and R¹¹ are independently hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the cyclic sulfonamide in Reaction Scheme B corresponds to Formula S-2 and the 1,3-diamine in Reaction Scheme B corresponds to Formula A-2:

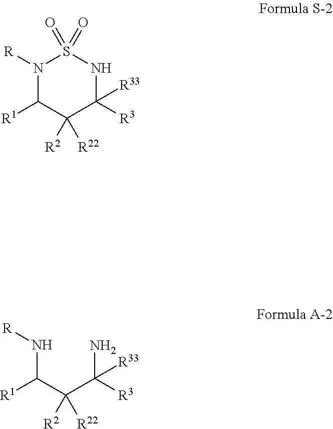

wherein R, R¹, R², R³, R²² and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the cyclic sulfonamide in Reaction Scheme B corresponds to Formula S-3 and the 1,3-diamine in Reaction Scheme B corresponds to Formula A-3:

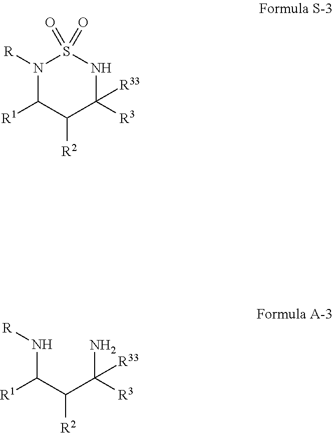

wherein R, R¹, R², R³, and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the cyclic sulfonamide in Reaction Scheme B corresponds to Formula S-4 and the 1,3-diamine in Reaction Scheme B corresponds to Formula A-4:

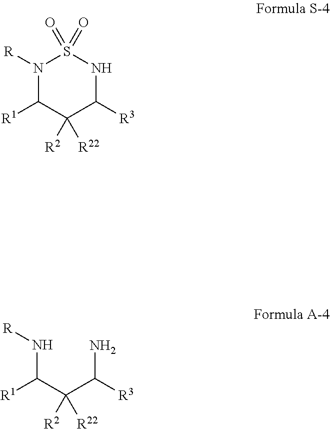

wherein R, R¹, R², R³, and R²² are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In one embodiment, the cyclic sulfonamide in Reaction Scheme B corresponds to Formula S-5 and the 1,3-diamine in Reaction Scheme B corresponds to Formula A-5:

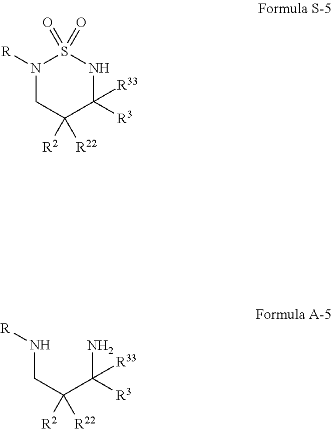

wherein R, R², R³, R²² and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the cyclic sulfonamide in Reaction Scheme B corresponds to Formula S-6 and the 1,3-diamine in Reaction Scheme B corresponds to Formula A-6:

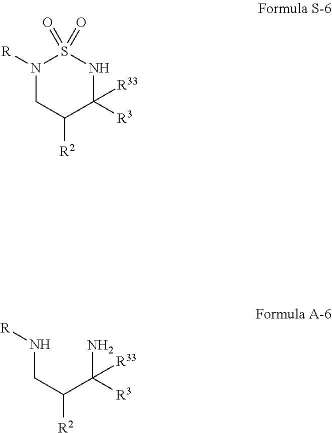

wherein R, R², R³, and R³³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo. By way of further example, in one such embodiment R³³ is hydrogen, hydrocarbyl, substituted hydrocarbyl, heterocyclo, hydrocarbyloxy, hydroxy, protected hydroxy, nitro, or acyl. By way of further example, in one such embodiment R³³ is hydrogen, hydrocarbyl, substituted hydrocarbyl, hydrocarbyloxy or heterocyclo. By way of further example, in one such embodiment R³ and R³³ are independently hydrogen, hydrocarbyl, substituted hydrocarbyl, hydrocarbyloxy or heterocyclo.

In one embodiment, the cyclic sulfonamide in Reaction Scheme B corresponds to Formula S-7 and the 1,3-diamine in Reaction Scheme B corresponds to Formula A-7:

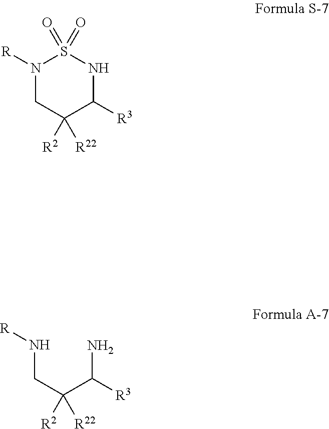

wherein R, R², R³, and R²² are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R²² is hydrogen, hydrocarbyl, substituted hydrocarbyl, heterocyclo, hydrocarbyloxy, hydroxy, protected hydroxy, nitro, or acyl. By way of further example, in one such embodiment R²² is hydrogen, hydrocarbyl, substituted hydrocarbyl, or heterocyclo. By way of further example, in one such embodiment R² and R²² are independently hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In one embodiment, the cyclic sulfonamide in Reaction Scheme B corresponds to Formula S-8 and the 1,3-diamine in Reaction Scheme B corresponds to Formula A-8:

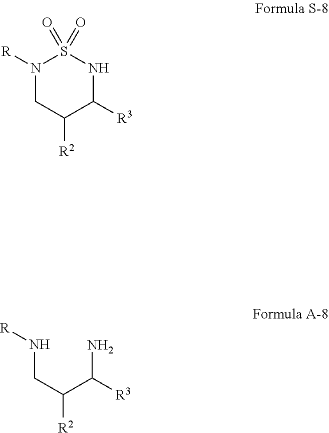

wherein R, R², and R³ are as previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

In one embodiment, the cyclic sulfonamide in Reaction Scheme B corresponds to Formula S-9 and the 1,3-diamine in Reaction Scheme B corresponds to Formula A-9:

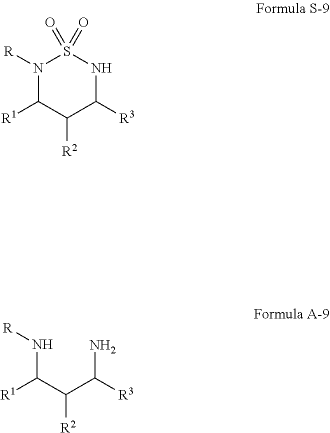

wherein R, R¹, R², and R³ areas previously defined in connection with Formula SA-1. For example, in one such embodiment R is hydrogen, hydrocarbyl, substituted hydrocarbyl, heterocyclo, or heterocyclo. By way of further example, in one such embodiment R is hydrocarbyl, substituted hydrocarbyl, or heterocyclo. By way of further example, in one such embodiment R¹ is hydrogen, hydrocarbyl, substituted hydrocarbyl, heterocyclo, hydrocarbyloxy, hydroxy, protected hydroxy, nitro, or acyl. By way of further example, in one such embodiment R¹ is hydrogen, hydrocarbyl, substituted hydrocarbyl, or heterocyclo. By way of further example, in one such embodiment R² is hydrogen, hydrocarbyl, substituted hydrocarbyl, heterocyclo, hydrocarbyloxy, hydroxy, protected hydroxy, nitro, or acyl. By way of further example, in one such embodiment R² is hydrogen, hydrocarbyl, substituted hydrocarbyl, or heterocyclo. By way of further example, in one such embodiment R³ is hydrogen, hydrocarbyl, substituted hydrocarbyl, heterocyclo, hydrocarbyloxy, hydroxy, protected hydroxy, nitro, or acyl. By way of further example, in one such embodiment R³ is hydrogen, hydrocarbyl, substituted hydrocarbyl, hydrocarbyloxy or heterocyclo.

Metal Porphyrins

The porphyrin with which the transition metal is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety)

In a preferred embodiment, the porphyrin is complexed with cobalt. The porphyrin with which cobalt is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In one embodiment, the metal porphyrin complex is a cobalt(II) porphyrin complex. In one particularly preferred embodiment, the cobalt porphyrin complex is a chiral porphyrin complex corresponding to the following structure

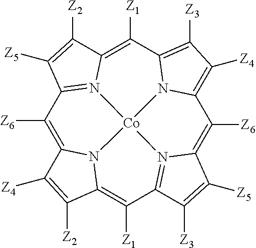

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, Z₆ is substituted phenyl, and Z₁ and Z₆ are different. In one particularly preferred embodiment, Z₂, Z₃, Z₄ and Z₅ are hydrogen, Z₁ is substituted phenyl, Z₆ is substituted phenyl, Z₁ and Z₆ are different, and the porphyrin is a chiral porphyrin. In one even further preferred embodiment, Z₂, Z₃, Z₄ and Z₅ are hydrogen, Z₁ is substituted phenyl, Z₆ is substituted phenyl, Z₁ and Z₆ are different and the porphyrin has D₂-symmetry.

In one embodiment, Z1 is selected from the group consisting of

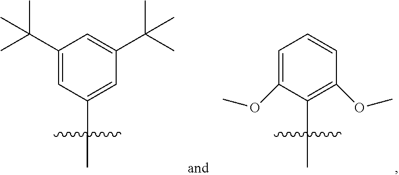

wherein

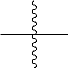

denotes the point of attachment to the porphyrin complex.

In one embodiment, Z₆ is selected from the group consisting of

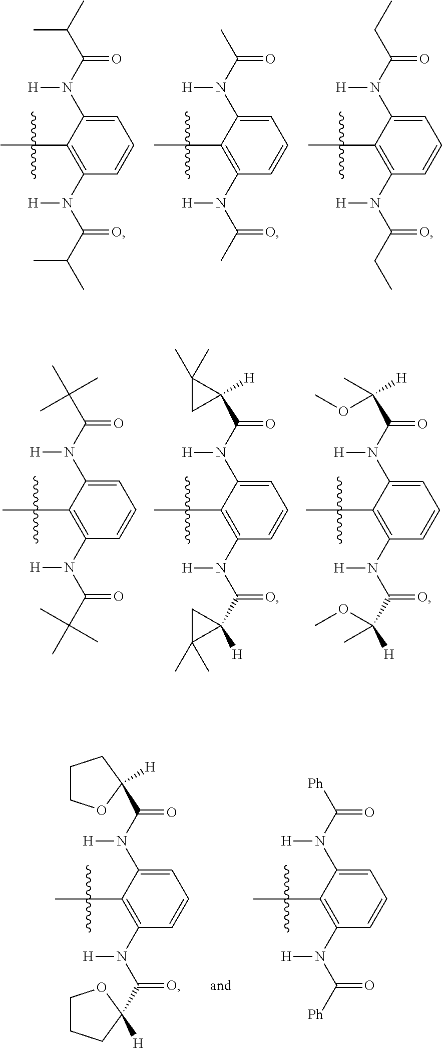

wherein

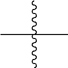

denotes the point of attachment to the porphyrin complex.

Exemplary cobalt(II) porphyrins include the following, designated [Co(P1)], [Co(P2)], [Co(P3)], [Co(P4)] and [Co(P5)].

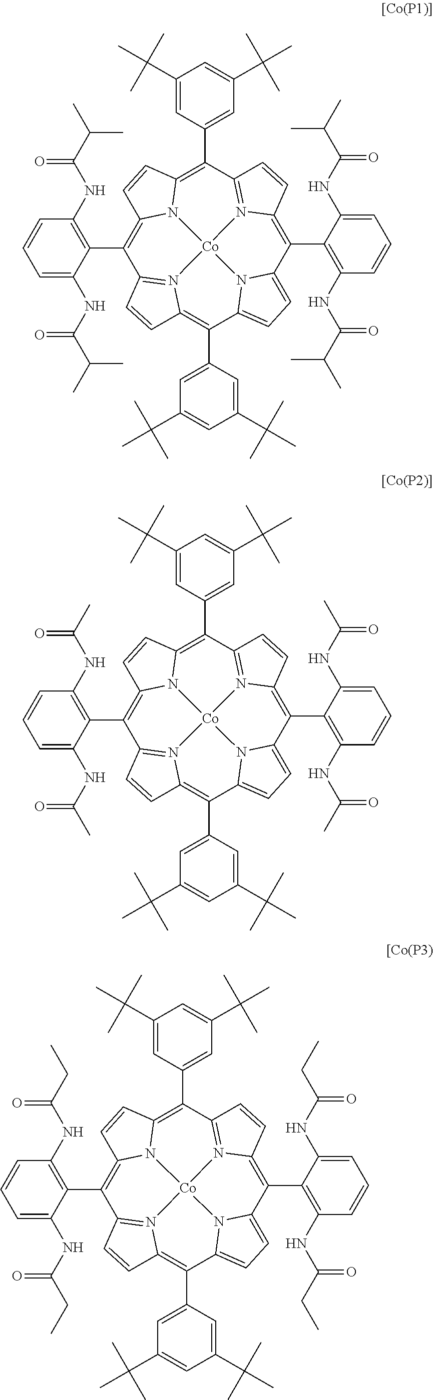

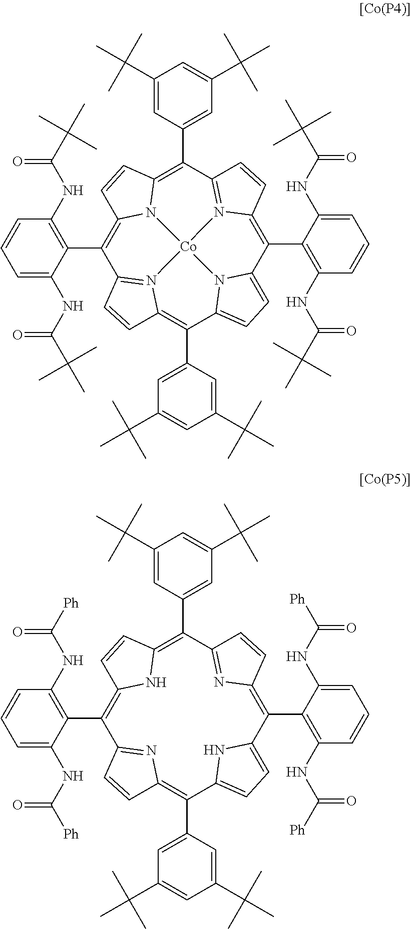

Other exemplary cobalt(II) porphyrins include the following, designated [Co(P11)], [Co(P12)], [Co(P13)], [Co(P14)], [Co(P15)], and [Co(P16)],

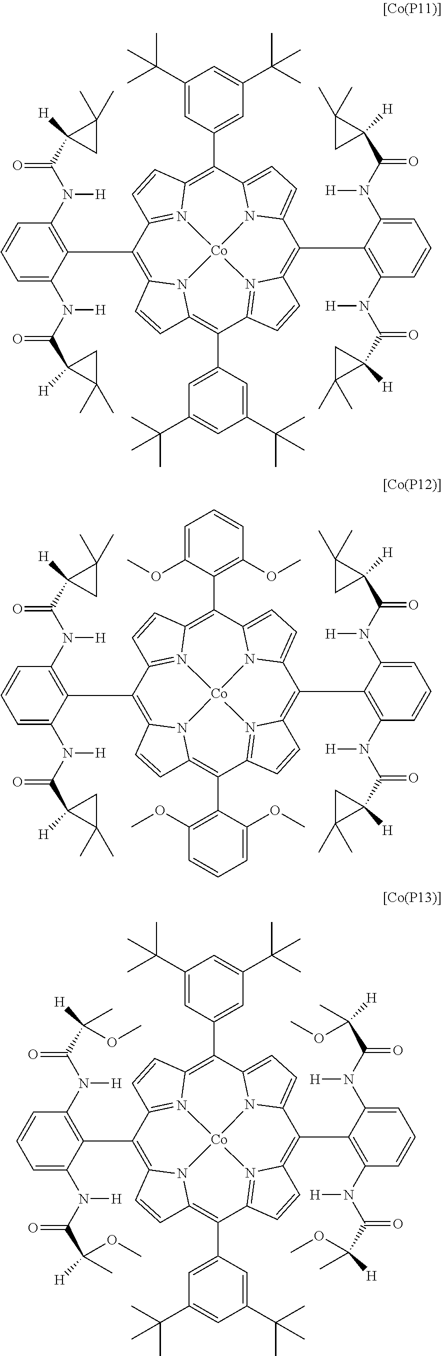

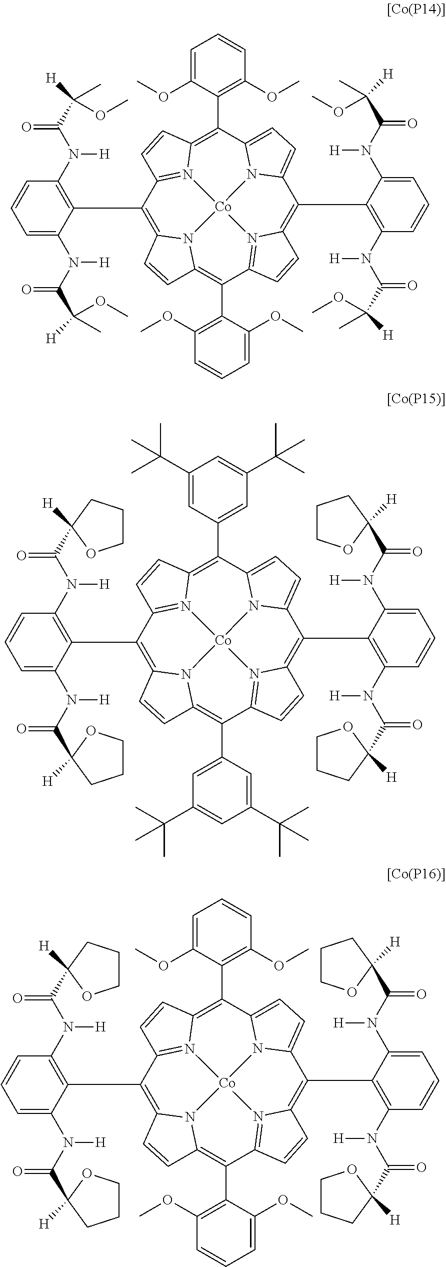

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

A wide range of sulfamoyl azides 2 could be conveniently prepared from corresponding amines 1 by following literature and its modified procedures (see Supplemental Information).^{12, 13} At the outset of this project, the simple azide 2a was selected as a model substrate for exploring the possibility of Co(II)-catalyzed intramolecular C—H amination of sulfamoyl azides and for establishing effective reaction conditions (Scheme 2). While the non-functionalized [Co(TPP)] was found to be an ineffective catalyst, [Co(P1)], in which the D₂ₕ-symmetric porphyrin 3,5-Di^{t}Bu-IbuPhyrin P1 has amide functionalities at the ortho-positions of the meso-phenyl groups, could effectively catalyze the intramolecular amination of 2a via a selective 1,6-C—H nitrene insertion process under mild conditions (Scheme 2). Due to the absence of oxidants or other additives, the Co(II)-catalyzed reaction was very clean, affording the desired 6-membered cyclic sulfonamide 3a as essentially the only product in 95% isolated yield (the byproduct was nitrogen gas). The difference in catalytic reactivity between [Co(TPP)] and [Co(P1)] can be considered as a dramatic demonstration of ligand-accelerated catalysis and could be rationalized as the outcome of a potential hydrogen-bonding interaction between the S═O and N—H units in the supposed nitrene intermediate.^{6b,14}

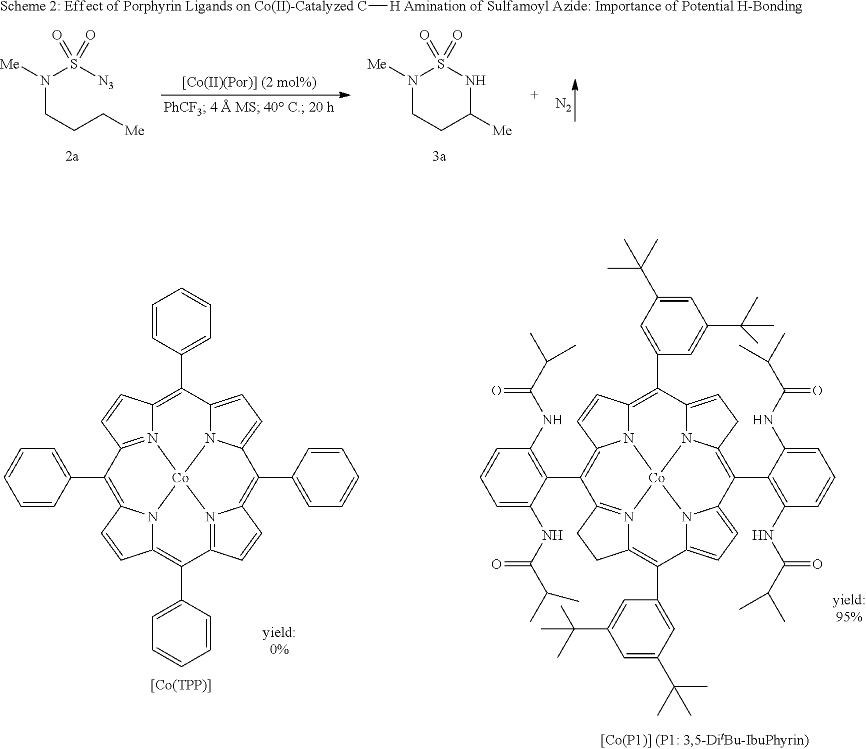

Under an optimized condition (2 mol % of [Co(P1)] in PhCF₃ at 40° C. for 20 h), the Co(II)-catalyzed C—H amination system was found to have a broad substrate scope and could be applied to a wide range of sulfamoyl azides (Table 1). In addition to the N-methyl group in 2a, the catalytic system could tolerate a variety of N-substituted functionalities due to its neutral and nonoxidative conditions, as demonstrated by the high-yielding amination reactions of azides 2b, 2c and 2d that contain ester, primary amide, and sulfide functional groups, respectively (entries 1-4). Predictably, benzylic C—H bonds could also be effectively aminated to provide the desired 6-membered cyclic sulfonamides in high yields (entries 5-6). The regioselectivity toward 1,6-C—H nitrene insertion could be best demonstrated with azide 2g, where only one of the five CH₂ units was selectively aminated to provide the corresponding cyclic sulfonamide 3g in 90% yield (entry 7).

TABLE 1
Catalytic Intramolecular Amination of Different Classes of
C—H Bonds via Activation of Sulfamoyl Azides by [Co(P1)].^{a}

| entry | sulfamoyl azide | cyclic sulfonamide | yield (%)^{b} |
|---|---|---|---|
| 1 | 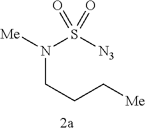 | 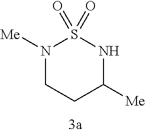 | 95% |
| 2 | 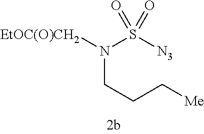 | 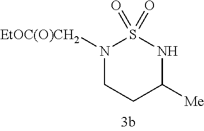 | 99% |
| 3^{c} | 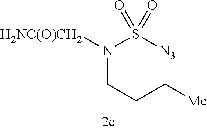 | 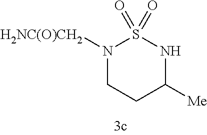 | 99% |
| 4^{d} | 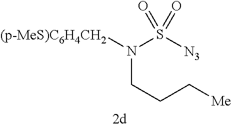 | 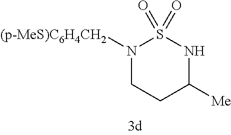 | 92% |
| 5 | 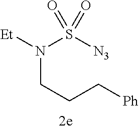 | 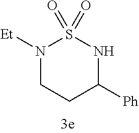 | 94% |
| 6 | 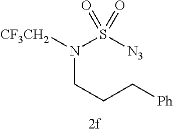 | 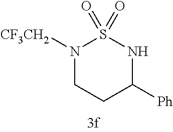 | 95% |
| 7^{e} | 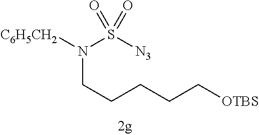 | 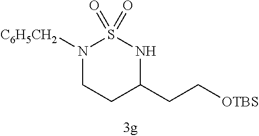 | 90% |
| 8 | 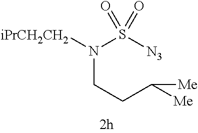 | 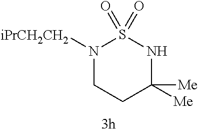 | 92% |
| 9^{c} | 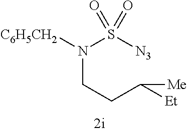 | 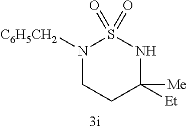 | 95% |
| 10^{c} | 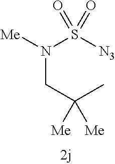 | 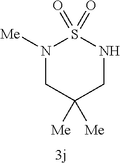 | 96% |
| 11^{c} | 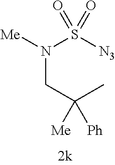 | 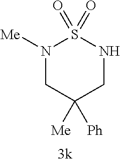 | 87% |
| 12^{c} | 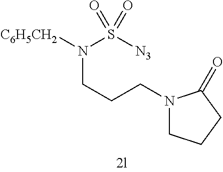 | 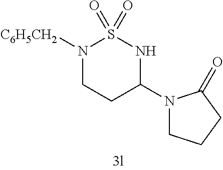 | 91% |
| 13 | 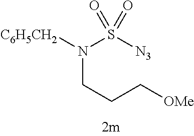 | 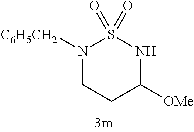 | >80%^{f} |

^{a}Performed in PhCF₃ at 40° C. for 20 h using 2 mol % [Co(P1)] under N₂ in the presence of 4 Å MS; [azide 2] = 0.10M.
^{b}Isolated yields.
^{c}In benzene.
^{d}In benzene at 80° C. for 3 h.
^{e}In benzene using 4 mol % [Co(P1)].
^{f}Estimated yield based on further transformation (see Scheme 3); the product was unstable for isolation

Besides various secondary C—H bonds, the Co(II)-catalyzed 1,6-C—H nitrene insertion process was shown to work equally well with tertiary C—H bonds as illustrated by the amination of sulfamoyl azides 2h and 2i (entries 8-9). Remarkably, even unactivated strong primary C—H bonds could be successfully aminated with this catalytic system, as nicely exemplified with the high-yielding reactions of substrates 2j and 2k (entries 10-11). Furthermore, the Co(II)-based catalytic system could be effectively utilized for intramolecular amination of heteroatom-activated C—H bonds such as the amidyl C—H bond in 2l and the ethereal C—H bond in 2m, providing the valuable N,N-acetal 3l and N,O-acetal 3m, respectively, in high yields (entries 12-13). To take advantage of the instability of N,O-acetals towards the formation of iminium ions, the amination product 3m could be directly transformed into other cyclic sulfonamides.¹⁵ As presented in Scheme 3, N,O-acetal 3m formed from amination of azide 2m could be in situ converted either to the unsubstituted cyclic sulfonamide 4m upon reduction with NaBH₄ or to the homoallyl-substituted cyclic sulfonamide 5m via reaction with allyl silane in the presence of BF₃.OEt₂. It is worth noting that these two-step transformations could be carried out in a one-pot protocol without any pre-workup of the amination reaction mixture due to the absence of oxidants and other additives in the catalytic system, further enhancing the practicality of these synthetic operations.

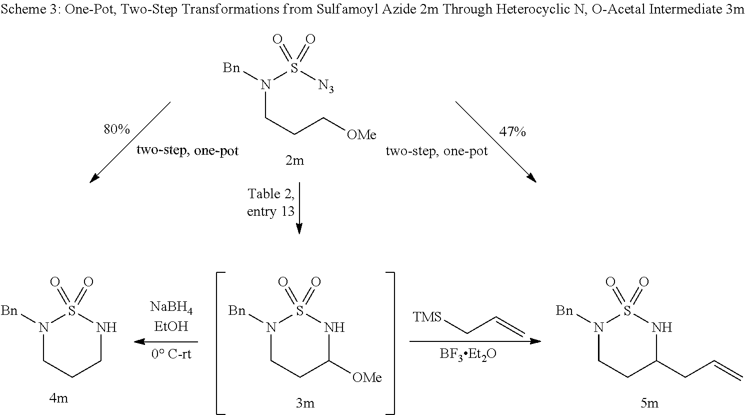

To assess diastereoselectivity of the [Co(P1)]/azide-based catalytic process, the C—H amination reaction of substrate 2n was carried out under standard conditions and furnished the desired cyclic sulfonamide 3n in 89% yield with a high diastereomeric ratio of 17 to 1 in favor of the trans isomer (eq 1). The relative configuration of trans-3n was further confirmed by X-ray crystallographic analysis (eq 1). When optically pure sulfamoyl azide (R)-2o was used as the substrate under similar conditions, the catalytic reaction by [Co(P1)] resulted in the high-yielding formation of the corresponding C—H amination product (R,R)-3o in excellent diastereoselectivity (eq 2).

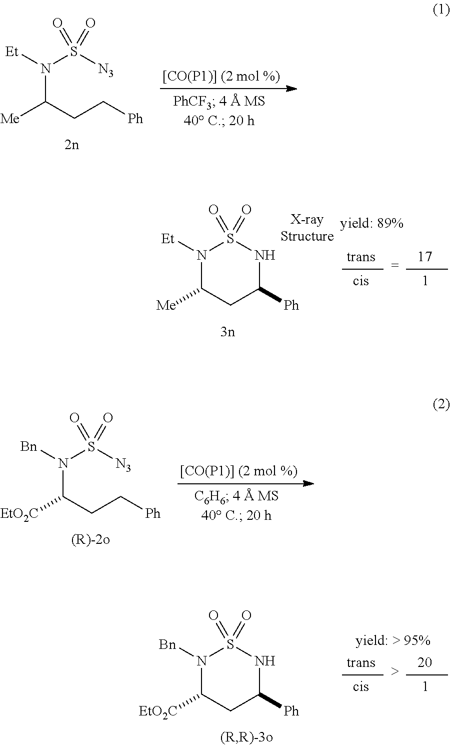

Further experiments were performed to evaluate the potential stereospecificity of the Co(II)-catalyzed C—H amination. To this end, enantiomerically pure sulfamoyl azide (S)-2p bearing a tertiary C—H bond was prepared and subjected to catalysis by [Co(P1)] (eq 3). Presumably due to the more demanding steric hindrance around the tertiary C—H bond and potential stabilization of the tertiary carbon radical intermediate by the phenyl group (vide infra), the resulting amination product 3p was obtained in a lower yield than the aforementioned tertiary C—H substrate 2h and 2i (Table 1, entries 8-9). Chiral HPLC analysis showed the cyclic sulfonamide (R)-3p maintained an enantiomeric excess of 85%, indicating partial racemization during catalysis (eq 3). When the optically pure azide (R)-2q was employed as the substrate instead, where the phenyl group at the tertiary carbon center of azide 2p was replaced by a alkyl group (more similar to substrates 2h and 2i; Table 1, entries 8-9), both high yield and high ee were obtained for the desired tertiary C—H amination product (S)-3q (eq 3). The absolute configuration of (S)-3q was further confirmed by X-ray crystallographic analysis (eq 4).

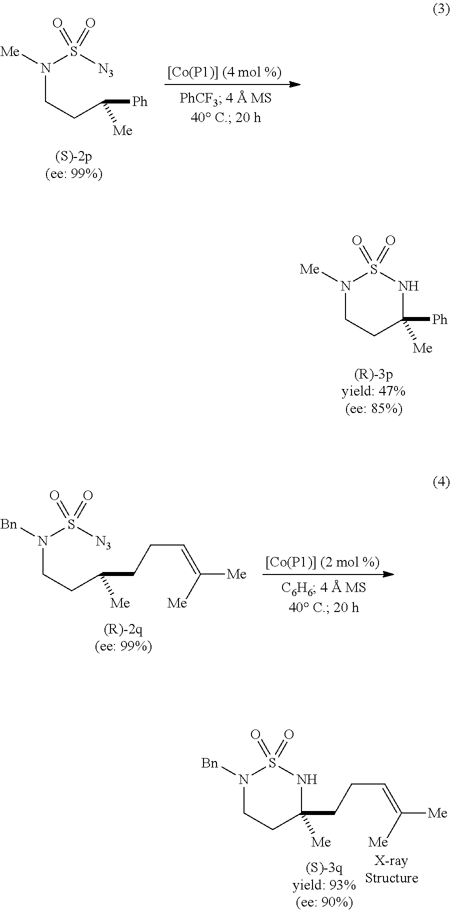

Taken together with the radical nature of the Co(II) catalyst, the above-observed reactivity and selectivity profile suggest the [Co(P1)]-catalyzed intramolecular C—H amination of sulfamoyl azides might operate with a mechanism that is different from the commonly-studied Rh₂-based amination system and may involve a ‘radical nitrene’ intermediate.^{6c} To shed more light on the suggested radical mechanism, sulfamoyl azide 2r, bearing a cyclopropyl unit, was designed and synthesized as a radical probe substrate for [Co(P1)]-catalyzed amination. As revealed in Scheme 4, the major product (85% yield) of the reaction is 6-membered cyclic sulfonamide 3r that resulted from 1,6-C—H nitrene insertion into the primary C—H bonds (˜100 Kcal/mol).¹⁶ Notably, the potential product 7r from amination of the more electron-rich but stronger secondary C—H (˜106 Kcal/mol)¹⁶ was not observed, indicating a key metallonitrene intermediate that performs radical H-atom abstraction rather than electrophilic C—H activation. More importantly, a small amount of 7-membered cyclic sulfonamide 6r was also generated (7% yield) from the catalytic reaction, a product that arose from cyclopropyl ring opening. It is proposed products 3r and 6r were derived from the same carbon radical species 2rB, which was generated from the key ‘radical nitrene’ intermediate 2rA via H-atom abstraction (Scheme 4).

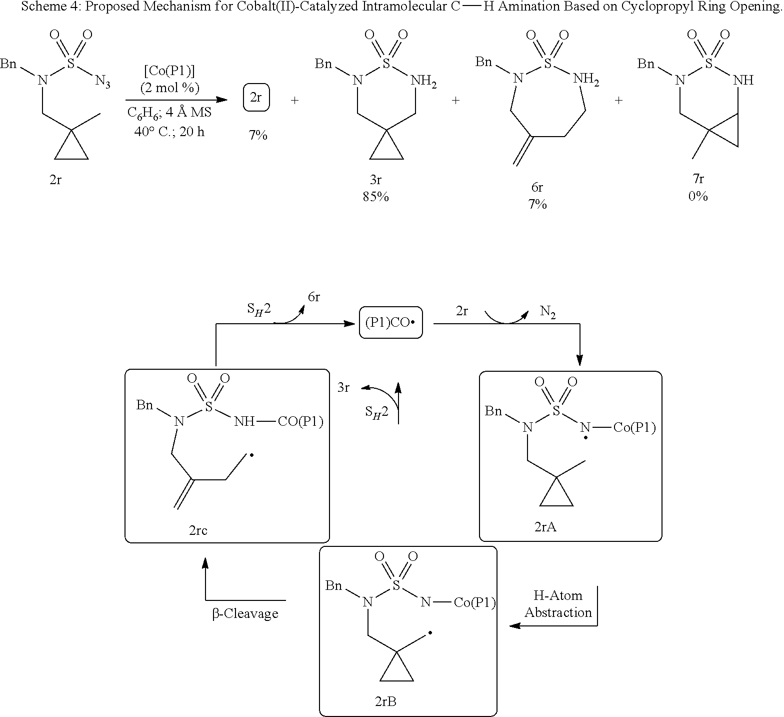

The series of cyclic sulfonamides 3 synthesized from the Co(II)-catalyzed C—H amination contains multifunctionalities and should find various potential applications, such as enzyme inhibitors in medicinal chemistry.¹⁷ Additionally, they can serve as convenient precursors for the preparation of valuable 1,3-diamine derivatives (Scheme 1). Among various methods, the SO₂ unit can be effectively removed under neutral conditions in refluxing 1,3-diaminopropane via transamination,¹⁸ as exemplified with the conversion of sulfamides 3i and 3q to the corresponding 1,3-diamines 8i and 8q, respectively (eqs 5 & 6).

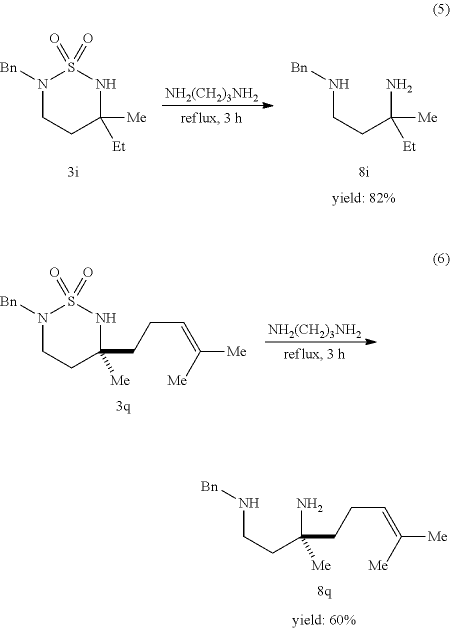

In summary, a Co(II)-based catalytic system has been developed for selective intramolecular C—H amination of a wide range of sulfamoyl azides under neutral and nonoxidative conditions. Together with the straightforward preparation of the azide substrates and the effective procedure for sulfone group deprotection, the high-yielding formation of cyclic sulfonamides via the Co(II)-catalyzed amination process offers a general access to valuable 1,3-diamine derivatives from corresponding monoamines. The catalytic system is highlighted with several salient features that are challenging problems for contemporary Rh₂-catalyzed amination, such as amination of strong primary C—H bonds and functional group tolerance. Fundamentally, the implied radical mechanism may have a far-reaching impact on our understanding and further development of catalytic C—H amination and related nitrene transfer processes.

### Supporting Information

General Considerations. All C—H amination reactions were performed under nitrogen in oven-dried glassware following standard Schlenk techniques. 4 Å molecular sieves were dried in a vacuum oven prior to use. Anhydrous C₆H₆ or PhCF₃ was purchased from Sigma-Aldrich and used without further purification. [Co(P1)] was prepared by following the literature.¹ Thin layer chromatography was performed on Merck TLC plates (silica gel 60 F254). Flash column chromatography was performed with ICN silica gel (60 Å, 230-400 mesh, 32-63 μm). ¹H NMR and ¹³C NMR were recorded on a Varian Inova400 (400 MHz) or a Bruker250 (250 MHz) instrument with chemical shifts reported relative to residual solvent. ¹⁹F NMR were recorded on a Varian Inova400 (400 MHz) instrument. Infrared spectra were measured with a Nicolet Avatar 320 spectrometer with a Smart Miracle accessory. HRMS data were obtained on an Agilent 1100 LC/MS/TOF mass spectrometer.

General Procedures for Synthesis of Sulfamoyl Azides

Method A:

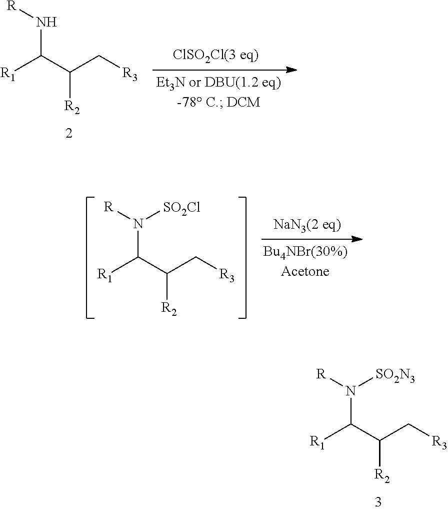

To a solution of SO₂Cl₂ (2 eq, 0.25 mol/L in DCM) at −78° C., a mixture of amine (1 eq) and base (1.2 eq) (Et₃N or DBU) in DCM was added dropwise via syringe. After stirring overnight at −78° C., the solution was warmed to 23° C. and stirred for 1 h. Solvent was removed under reduced pressure at room temperature. Purification of the resulting mixture by flash column chromatography using silica gel (10:1 hexane/ethyl acetate) afforded the sulfamoyl chlorides.

Sodium azide (2.0 eq) and Bu₄NBr (0.3 eq) was added in portions to the sulfamoyl chloride in acetone (0.2 mol/L) and the reaction was monitored by crude ¹H NMR until completion (typically 12 hrs). After the reaction was completed, the flask underwent rotary evaporation at room temperature until most of the acetone was removed. The reaction mixture was purified by flash chromatography. The fractions containing product were collected and concentrated by rotary evaporation at room temperature to afford the azide. Note: Some azides could be explosive and should be handled carefully. Based on DSC experiments (see Page 21, DSC spectrogram of azide 2e), this type of azide is stable under reaction conditions used.

Method B:

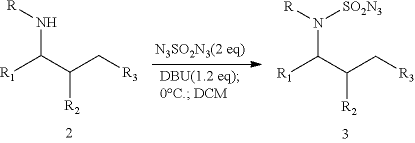

Sulphuryl Azide.² To a solution of sodium azide (2.6 g, 40 mmol) and pyridine (1.58 g, 20 mmol) in acetonitrile (50 ml) at 0° C., sulphuryl chloride (1.34 g, 10 mmol) in acetonitrile (20 ml) was added dropwise for 10 min. Then the reaction mixture was stirred for a further 1 h at room temperature. After addition of 30 ml DCM, the mixture was poured into ice-cold water and extracted with DCM (3×20 mL) The combined organic layer was washed with hydrochloric acid (1 mol/L in H₂O), water, potassium hydroxide (1 mol/L in H₂O), hydrochloric acid (1 mol/L in H₂O), and water. After drying (Na₂SO₄), the sulphuryl azide solution was used directly for the subsequent reaction. This solution can be stored in the refrigerator for at least two months without significant decomposition.

To a solution of N₃SO₂N₃ (2 eq, 0.25 mol/L in DCM) at 0° C., a mixture of amine (1 eq) and DBU (1.2 eq) in DCM was added dropwise via syringe. The reaction showed almost complete consumption of the starting amine after 5 min to 3 hours when monitored by TLC, then the majority of the solvent was removed under reduced pressure at room temperature. Purification of this mixture by chromatography on silica gel (as given below) afforded the sulfamoyl azide. Note: Some azides could be explosive and should be handled carefully. Based on DSC experiments (see Page 21, DSC spectrogram of azide 2e), this type of azide is stable under the reaction conditions used.

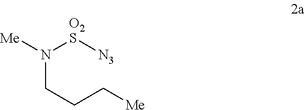

Prepared according to METHOD A (yield 65%; 2 steps). Purified by chromatography on silica gel (20:1 hexanes/EtOAc), colorless liquid, TLC R_{f}=0.52 (10:1 hexanes/EtOAc). ¹H NMR (250 MHz, CDCl₃): δ 3.20 (t, J=7.3 Hz, 2H), 2.90 (s, 3H), 1.63-1.50 (m, 2H), 1.40-1.27 (m, 2H), 0.92 (t, J=7.3 Hz, 3H). ¹³C NMR (62.9 MHz, CDCl₃): δ 51.1, 35.6, 29.1, 19.5, 13.5. IR (neat, cm⁻¹): 2962, 2124, 1386, 1207, 1162, 932, 758, 735.

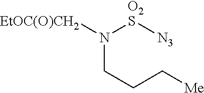

Prepared according to METHOD A (yield 48%; 2 steps). Purified by chromatography on silica gel (10:1 hexanes/EtOAc), colorless liquid, TLC R_{f}=0.35 (10:1 hexanes/EtOAc). ¹H NMR (250 MHz, CDCl₃): δ 4.23 (q, J=7.0 Hz, 2H), 4.07 (s, 2H), 3.38 (t, J=7.5 Hz, 2H), 1.63-1.50 (m, 2H), 1.38-1.31 (m, 2H), 1.28 (t, J=7.0 Hz, 3H), 0.91 (t, J=7.3 Hz, 3H). ¹³C NMR (62.9 MHz, CDCl₃): δ 168.3, 61.9, 49.5, 49.3, 29.6, 19.6, 14.1, 13.6. IR (neat, cm⁻¹): 2963, 2129, 1748, 1382, 1212, 1184, 1162, 1136, 1058, 935, 773, 738.

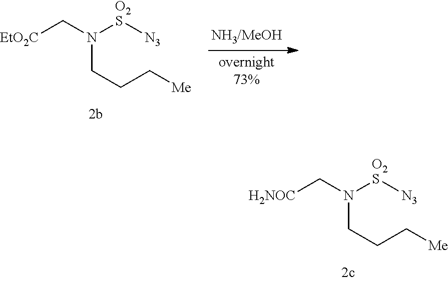

Azide 2b (260 mg, 1.0 mmol) was added to a solution of NH₃ (2 ml, 7N in MeOH). The reaction was stirred overnight at room temperature. Most of the solvent was removed under reduced pressure at room temperature, then purified by chromatography on silica gel (gradient elution: 1:1-1:2 hexanes/EtOAc), colorless liquid, TLC R_{f}=0.17 (1:1 hexanes/EtOAc). ¹H NMR (250 MHz, CDCl₃): δ 6.14 (brs, 1H), 5.78 (brs, 1H), 3.90 (s, 2H), 3.36 (t, J=7.8 Hz, 2H), 1.65-1.54 (m, 2H), 1.41-1.25 (m, 2H), 0.93 (t, J=7.3 Hz, 3H). ¹³C NMR (62.9 MHz, CDCl₃): δ 169.5, 51.7, 51.0, 29.5, 19.7, 13.6. IR (neat, cm⁻¹): 2129, 1678, 1371, 1158, 1052, 929, 780.736.

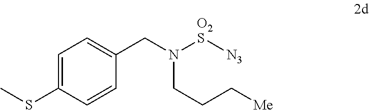

Prepared according to METHOD B (yield 90%). Purified by chromatography on silica gel (20:1 hexanes/EtOAc), colorless liquid, TLC R_{f}=0.42 (10:1 hexanes/EtOAc). ¹H NMR (400 MHz, CDCl₃): δ 7.23 (brs, 4H), 4.38 (s, 2H), 3.17 (t, J=8.0 Hz, 2H), 2.46 (s, 3H), 1.56-1.47 (m, 2H), 1.29-1.19 (m, 2H), 0.86 (t, J=7.2 Hz, 3H). ¹³C NMR (100 MHz, CDCl₃): δ 139.0, 131.3, 129.0, 126.6, 51.8, 48.2, 29.2, 19.7, 15.6, 13.5. IR (neat, cm⁻¹): 2122, 1378, 1162, 1095, 1035, 916, 769, 733.

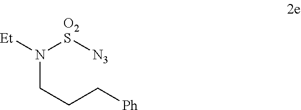

Prepared according to METHOD A (yield 51%; 2 steps). Purified by chromatography on silica gel (20:1 hexanes/EtOAc), colorless liquid, TLC R_{f}=0.50 (10:1 hexanes/EtOAc). ¹H NMR (400 MHz, CDCl₃): δ 7.31-7.16 (m, 5H), 3.33 (q, J=7.2 Hz, 2H), 3.28 (t, J=7.6 Hz, 2H), 2.65 (t, J=7.6 Hz, 2H), 1.99-1.91 (m, 2H), 1.20 (t, J=7.2 Hz, 3H). ¹³C NMR (100 MHz, CDCl₃): δ 140.7, 128.5, 128.2, 126.2, 48.2, 44.1, 32.7, 29.6, 13.2. IR (neat, cm⁻¹): 2121, 1379, 1200, 1159, 1004, 735, 698.

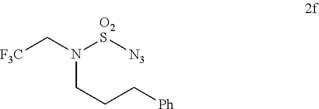

Prepared according to METHOD B (yield 60%; based on 70% conversion). Purified by chromatography on silica gel (20:1 hexanes/EtOAc), colorless liquid, TLC R_{f}=0.42 (10:1 hexanes/EtOAc). ¹H NMR (400 MHz, CDCl₃): δ 7.33-7.14 (m, 5H), 3.84 (q, J=8.5 Hz, 2H), 3.43 (t, J=7.8 Hz, 2H), 2.64 (t, J=7.5 Hz, 2H), 2.08-1.95 (m, 2H). ¹³C NMR (62.9 MHz, CDCl₃): δ 140.2, 128.7, 128.2, 126.4, 123.4 (q, J=280 Hz), 50.3, 49.5 (q, J=35 Hz), 32.6, 28.9. ¹⁹F NMR (377 MHz, CDCl₃): δ=−70.6 (t, J=8.3 Hz). IR (neat, cm⁻¹): 2134, 1389, 1274, 1166, 1088, 1014, 780, 745, 665.

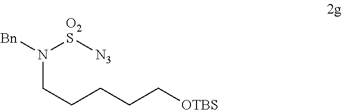

Prepared according to METHOD A (yield 40%; 2 steps) or METHOD B (yield 75%). Purified by chromatography on silica gel (20:1 hexanes/EtOAc), colorless liquid, TLC R_{f}=0.57 (10:1 hexanes/EtOAc). ¹H NMR (250 MHz, CDCl₃): δ 7.36-7.31 (m, 5H), 4.34 (s, 2H), 3.53 (t, J=6.3 Hz, 2H), 3.19 (t, J=7.5 Hz, 2H), 1.62-1.38 (m, 4H), 1.31-1.19 (m, 2H), 0.87 (s, 9H), 0.01 (s, 6H). ¹³C NMR (62.9 MHz, CDCl₃): δ 134.7, 128.8, 128.5, 128.4, 62.7, 52.4, 48.6, 32.2, 27.0, 25.9, 22.8, 18.3, −5.3. IR (neat, cm⁻¹): 2930, 2123, 1384, 1255, 1207, 1167, 1099, 834, 774, 735, 698.

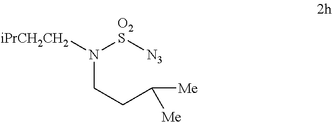

Prepared according to METHOD A (yield 47% 2 steps). Purified by chromatography on silica gel (20:1 hexanes/EtOAc), colorless liquid, TLC R_{f}=0.50 (10:1 hexanes/EtOAc). ¹H NMR (250 MHz, CDCl₃): δ 3.28-3.20 (m, 4H), 1.65-1.43 (m, 6H), 0.91 (d, J=6.3 Hz, 12H). ¹³C NMR (62.9 MHz, CDCl₃): δ 47.5, 36.6, 25.8, 22.4. IR (neat, cm⁻¹): 2960, 2120, 1468, 1384, 1206, 1180, 1157, 931, 731.

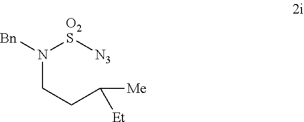

Prepared according to METHOD B (yield 95%). Purified by chromatography on silica gel (20:1 hexanes/EtOAc), colorless liquid, TLC R_{f}=0.44 (10:1 hexanes/EtOAc). ¹H NMR (250 MHz, CDCl₃): δ 7.34 (s, 5H), 4.43 (s, 2H), 3.24-3.17 (m, 2H), 1.64-1.50 (m, 1H), 1.38-1.03 (m, 4H), 0.80 (t, J=7.3 Hz, 3H), 0.79 (d, J=6.3 Hz, 3H). ¹³C NMR (62.9 MHz, CDCl₃): δ 134.6, 128.8, 128.5, 128.4, 52.2, 46.8, 33.5, 32.0, 29.1, 18.8, 11.1. IR (neat, cm⁻¹): 2122, 1457, 1380, 1206, 1165, 1051, 934, 735, 698.

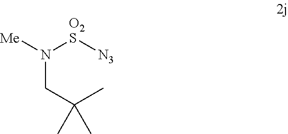

Prepared according to METHOD A (yield 55%; 2 steps). Purified by chromatography on silica gel (20:1 hexanes/EtOAc), colorless liquid, TLC R_{f}=0.45 (10:1 hexanes/EtOAc). ¹H NMR (250 MHz, CDCl₃): δ 3.00 (s, 3H), 2.96 (s, 2H), 0.96 (s, 9H). ¹³C NMR (62.9 MHz, CDCl₃): δ 63.5, 39.9, 33.3, 27.8. IR (neat, cm⁻¹): 2963, 2123, 1380, 1367, 1200, 1165, 979, 764, 746.

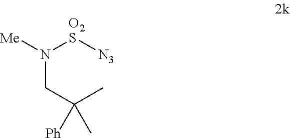

Prepared according to METHOD B (yield 91%). Purified by chromatography on silica gel (20:1 hexanes/EtOAc), colorless liquid, TLC R_{f}=0.52 (10:1 hexanes/EtOAc). ¹H NMR (400 MHz, CDCl₃): δ 7.40-7.21 (m, 5H), 3.32 (s, 2H), 2.39 (s, 3H), 1.42 (s, 6H). ¹³C NMR (100 MHz, CDCl₃): δ 146.1, 128.5, 126.6, 126.0, 63.8, 39.1, 38.5, 26.4. IR (neat, cm⁻¹): 2123, 1379, 1201, 1165, 986, 959, 759, 700.

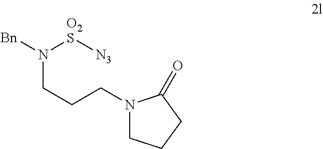

Prepared according to METHOD B (yield 80%). Purified by chromatography on silica gel (gradient elution: 2:1-1:3 hexanes/EtOAc), colorless liquid (white solid at −20° C.), TLC R_{f}=0.21 (1:2 hexanes/EtOAc). ¹H NMR (250 MHz, CDCl₃): 7.33 (s, 5H), 4.41 (s, 2H), 3.19-3.12 (m, 6H), 2.29 (t, J=8.0 Hz, 2H), 1.98-1.85 (m, 2H), 1.76-1.63 (m, 2H). ¹³C NMR (62.9 MHz, CDCl₃): 175.1, 134.4, 128.9, 128.5, 53.2, 46.7, 46.5, 39.5, 30.8, 25.6, 17.8. IR (neat, cm⁻¹): 2125, 1679, 1377, 1262, 1207, 1164, 1021, 795, 737, 700.

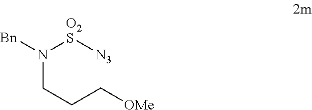

Prepared according to METHOD A (yield 58%; 2 steps) or METHOD B (yield 88%). Purified by chromatography on silica gel (gradient elution: 20:1-10:1 hexanes/EtOAc), colorless liquid, TLC R_{f}=0.27 (10:1 hexanes/EtOAc). ¹H NMR (250 MHz, CDCl₃): δ 7.37-7.30 (m, 5H), 4.44 (s, 2H), 3.36-3.28 (m, 4H), 3.24 (s, 3H), 1.85-1.74 (m, 2H). ¹³C NMR (62.9 MHz, CDCl₃): δ 134.7, 128.8, 128.5, 128.4, 69.2, 58.5, 52.9, 46.1, 27.8. IR (neat, cm⁻¹): 2124, 1378, 1196, 1165, 1114, 786, 735, 698.

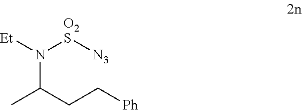

Prepared according to METHOD A (yield 50%; 2 steps). Purified by chromatography on silica gel (gradient elution: 50:1-30:1 hexanes/EtOAc), colorless liquid, TLC R_{f}=0.72 (30:1 hexanes/EtOAc). ¹H NMR (250 MHz, CDCl₃): δ 7.25-7.09 (m, 5H), 3.91-3.81 (m, 1H), 3.20 (q, J=7.0 Hz, 2H), 2.66-2.57 (m, 2H), 1.95-1.63 (m, 2H), 1.23 (t, J=7.0 Hz, 3H), 1.22 (d, J=6.5 Hz, 3H). ¹³C NMR (62.9 MHz, CDCl₃): δ 141.0, 128.5, 128.2, 126.1, 56.1, 40.0, 36.9, 32.8, 18.7, 15.9. IR (neat, cm⁻¹): 2119, 1373, 1206, 1181, 1163, 1136, 732, 699.

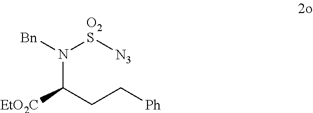

Prepared according to METHOD A (yield 13%; 2 steps). Purified by chromatography on silica gel (10:1 hexanes/EtOAc), colorless liquid, TLC R_{f}=0.41 (10:1 hexanes/EtOAc). ¹H NMR (400 MHz, CDCl₃): δ 7.41-7.32 (m, 5H), 7.21-7.13 (m, 3H), 6.86 (d, J=6.8 Hz, 2H), 4.75, 4.43 (AB q, J=16.0 Hz, each 1H), 4.46-4.41 (m, 1H), 4.19-4.12 (m, 2H), 2.53-2.47 (m, 2H), 2.22-2.12 (m, 1H), 1.97-1.87 (m, 1H), 1.26 (t, J=7.2 Hz, 3H). ¹³C NMR (62.9 MHz, CDCl₃): δ 169.9, 140.1, 135.7, 128.7, 128.6, 128.4, 128.3, 126.2, 62.0, 61.7, 51.4, 32.4, 31.7, 14.0. IR (neat, cm⁻¹): 2129, 1738, 1388, 1204, 1164, 1028, 745, 698.

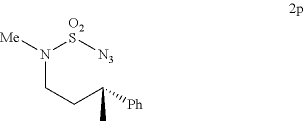

Prepared according to METHOD A (yield 54%; 2 steps) or METHOD B (yield 89%). Purified by chromatography on silica gel (20:1 hexanes/EtOAc), colorless liquid, TLC R_{f}=0.43 (10:1 hexanes/EtOAc). ¹H NMR (250 MHz, CDCl₃): δ 7.27-7.20 (m, 2H), 7.16-7.09 (m, 3H), 3.18-2.92 (m, 2H), 2.78 (s, 3H), 2.73-2.63 (m, 1H), 1.88-1.78 (m, 2H), 1.21 (d, J=6.8 Hz, 3H). ¹³C NMR (62.9 MHz, CDCl₃): δ 145.7, 128.6, 126.7, 126.4, 50.0, 37.2, 35.7, 35.2, 22.3. IR (neat, cm⁻¹): 2123, 1453, 1378, 1205, 1164, 954, 759, 734, 700. HPLC analysis: ee>99%. Chiralcel OD-H (95% hexanes: 5% isopropanol, 0.8 mL/min), tₘᵢₙₒᵣ=8.6 min, tₘₐⱼₒᵣ=9.7 min.

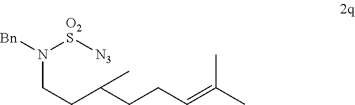

Prepared according to METHOD B (yield 88%). Purified by chromatography on silica gel (gradient elution: 20:1-10:1 hexanes/EtOAc), colorless liquid, TLC R_{f}=0.35 (10:1 hexanes/EtOAc). ¹H NMR (250 MHz, CDCl₃): δ 7.34 (s, 5H), 5.05-4.97 (m, 1H), 4.42 (s, 2H), 3.20 (t, J=8.0 Hz, 2H), 1.95-1.83 (m, 2H), 1.65 (d, J=0.8 Hz, 3H), 1.62-1.50 (m, 1H), 1.56 (s, 3H), 1.39-1.05 (m, 4H), 0.80 (d, J=6.3 Hz, 3H). ¹³C NMR (62.9 MHz, CDCl₃): δ 134.7, 131.5, 128.8, 128.5, 128.4, 124.3, 52.2, 46.8, 36.7, 33.9, 30.0, 25.7, 25.2, 19.2, 17.6. IR (neat, cm⁻¹): 2123, 1456, 1380, 1206, 1166, 735, 698. The ee was determinated by corresponding starting material N-benzyl-3,7-dimethyloct-6-enamide: HPLC analysis: ee>99%. Chiralcel OD-H (98% hexanes: 2% isopropanol, 0.8 mL/min), tₘᵢₙₒᵣ=76.2 min, tₘₐⱼₒᵣ=85.1 min.

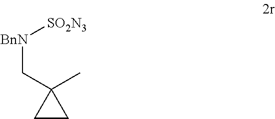

Prepared according to METHOD B (yield 70%). Purified by chromatography on silica gel (gradient elution: 30:1-20:1 hexanes/EtOAc), colorless liquid, TLC R_{f}=0.53 (10:1 hexanes/EtOAc). ¹H NMR (400 MHz, CDCl₃): δ 7.38-7.28 (m, 5H), 4.60 (s, 2H), 3.17 (s, 2H), 1.13 (s, 3H), 0.33 (d, J=3.2 Hz, 2H), 0.31 (d, J=3.2 Hz, 2H). ¹³C NMR (100 MHz, CDCl₃): δ 134.8, 128.8, 128.1, 56.5, 52.1, 21.1, 13.8, 11.8. IR (neat, cm⁻¹): 2122, 1377, 1204, 1165, 1066, 1028, 937, 887, 776, 735, 697.

### General Procedure for C—H Amination of Sulfamoyl Azides

An oven dried Schlenk tube was charged with catalyst (0.002 mmol) and 4 Å MS (50 mg), then evacuated and back filled with nitrogen. The Teflon screw cap was replaced with a rubber septum and then an approximately 0.5 ml portion of benzene (or PhCF₃) was added, then azide (0.1 mmol), followed by the remaining benzene (or PhCF₃) (total 1 mL). The Schlenk tube was then purged with nitrogen for 2 minutes and the rubber septum was replaced with a Teflon screw cap. The Schlenk tube was then placed in an oil bath for the desired time and temperature. After completion of the reaction, the reaction mixture was purified by flash column chromatography. The fractions containing product were collected and concentrated by rotary evaporation to afford the target compound.

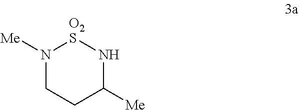

Purified by chromatography on silica gel (gradient elution: 2:1-1:1 hexanes/EtOAc), white solid, TLC R_{f}=0.56 (1:1 hexanes/EtOAc). ¹H NMR (400 MHz, CDCl₃): δ 3.91 (d, J=9.5 Hz, 1H), 3.74-3.58 (m, 1H), 3.42-3.29 (m, 1H), 3.11-3.02 (m, 1H), 2.68 (s, 3H), 1.62-1.52 (m, 2H), 1.17 (d, J=6.5 Hz, 3H). ¹³C NMR (62.9 MHz, CDCl₃): δ 52.5, 52.0, 36.3, 30.9, 21.2. IR (neat, cm⁻¹): 2922, 1331, 1299, 1159, 1131, 1078, 1024, 858, 756, 617. HRMS (ESI) [M+H]⁺) Calcd. for C₅H₁₃N₂O₂S 165.0692. Found 165.0690.

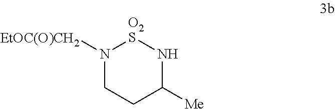

Purified by chromatography on silica gel (1:1 hexanes/EtOAc), white solid, TLC R_{f}=0.27 (1:1 hexanes/EtOAc). ¹H NMR (250 MHz, CDCl₃): δ 4.30, 3.66 (AB q, J=17.8 Hz, each 1H), 4.33-4.21 (m, 3H), 3.95-3.67 (m, 2H), 3.44-3.35 (m, 1H), 1.88-1.66 (m, 2H), 1.36 (t, J=7.3 Hz, 3H), 1.33 (d, J=6.5 Hz, 3H). ¹³C NMR (250 MHz, CDCl₃): δ 169.7, 61.6, 52.9, 50.7, 50.2, 30.5, 21.4, 14.1. IR (neat, cm⁻¹): 1738, 1421, 1347, 1308, 1207, 1165, 1138, 1114, 857, 762, 618. HRMS (ESI) ([M+H]⁺) Calcd. for C₈H₁₇N₂O₄S 237.0904. Found 237.0899.

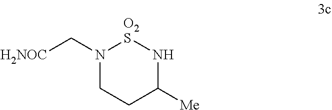

Purified by chromatography on silica gel (gradient elution: 15:1-10:1 DCM/MeOH), white solid, TLC R_{f}=0.35 (10:1 DCM/MeOH); ¹H NMR (250 MHz, (CD₃)₂SO): δ 7.29 (d, J=12.8 Hz, 2H), 6.61 (brs, 1H), 3.61, 3.36 (AB q, J=16.5 Hz, each 1H), 3.36-3.23 (m, 3H), 1.53-1.43 (m, 2H), 1.10 (t, J=6.5 Hz, 3H). ¹³C NMR (62.9 MHz, (CD₃)₂SO): δ 170.0, 52.0, 50.5, 50.3, 28.7, 20.6. IR (neat, cm⁻¹): 2921, 1654, 1409, 1353, 1163, 1077, 751, 668. HRMS (ESI) ([M+Na]⁺) Calcd. for C₆H₁₃N₃O₃SNa 230.0570. Found 230.0571.

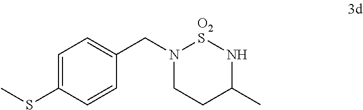

Purified by chromatography on silica gel (4:1 hexanes/EtOAc), white solid, TLC R_{f}=0.69 (1:1 hexanes/EtOAc). ¹H NMR (250 MHz, CDCl₃): δ 7.26-7.17 (m, 4H), 4.47, 3.80 (AB q, J=13.8 Hz, each 1H), 3.87-3.69 (m, 2H), 3.22 (dt, J=3.0, 12.8 Hz, 1H), 3.08-2.99 (m, 1H), 2.46 (s, 3H), 1.66-1.36 (m, 2H), 1.22 (d, J=6.3 Hz, 3H). ¹³C NMR (62.9 MHz, CDCl₃): δ 138.3, 132.1, 129.3, 126.6, 52.5, 51.3, 47.6, 31.5, 21.3, 15.7. IR (neat, cm⁻¹): 3270, 2923, 1494, 1408, 1330, 1305, 1166, 1134, 1095, 862, 750, 708. HRMS (ESI) ([M+H]⁺) Calcd. For C₁₂H₁₉N₂O₂S₂ 287.0883. Found 287.0877.

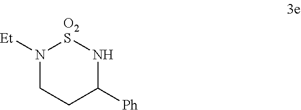

Purified by chromatography on silica gel (gradient elution: 4:1-2:1 hexanes/EtOAc), white solid, TLC R_{f}=0.21 (4:1 hexanes/EtOAc). ¹H NMR (250 MHz, CDCl₃): δ 7.38-7.30 (m, 5H), 4.78-4.68 (m, 1H), 4.07 (d, J=7.8 Hz, 1H), 3.68-3.55 (m, 1H), 3.41-3.15 (m, 3H), 2.10-1.86 (m, 2H), 1.22 (t, J=7.0 Hz, 3H). ¹³C NMR (62.9 MHz, CDCl₃): δ 139.3, 128.9, 128.4, 126.3, 59.6, 47.9, 43.5, 29.5, 13.1. IR (neat, cm⁻¹): 2980, 1262, 1160, 1147, 1091, 1059, 1036, 825, 803, 753. HRMS (ESI) ([M+Na]⁺) Calcd. for C₁₁H₁₆N₂O₂SNa 263.0825. Found 263.0829.

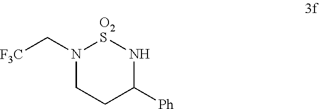

Purified by chromatography on silica gel (4:1 hexanes/EtOAc), white solid, TLC R_{f}=0.27 (4:1 hexanes/EtOAc). ¹H NMR (250 MHz, CDCl₃): δ 7.40-7.30 (m, 5H), 4.82-4.72 (m, 1H), 4.26 (d, J=7.3 Hz, 1H), 3.85-3.47 (m, 4H), 2.15-1.90 (m, 2H). ¹³C NMR (62.9 MHz, CDCl₃): δ 138.6, 129.1, 128.8, 126.2, 121.8, 59.8, 50.6, 49.2 (q, J=34 Hz), 29.4. ¹⁹F NMR (377 MHz, CDCl₃): δ=−71.7 (t, J=8.7 Hz). IR (neat, cm⁻¹): 3235, 2925, 1456, 1339, 1279, 1163, 1150, 1072, 916, 770, 756, 720, 697. HRMS (ESI) ([M+Na]⁺) Calcd. for C₁₁H₁₄N₂O₂F₃S 295.0723. Found 295.0729.

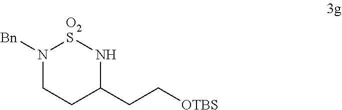

Purified by chromatography on silica gel (4:1 hexanes/EtOAc), white solid, TLC R_{f}=0.31 (4:1 hexanes/EtOAc). ¹H NMR (250 MHz, CDCl₃): δ 7.34-7.28 (m, 5H), 4.87 (d, J=8.8 Hz, 1H), 4.49, 3.90 (AB q, J=14.0 Hz, each 1H), 3.87-3.71 (m, 3H), 3.29 (dt, J=3.0, 12.8 Hz, 1H), 3.11-3.02 (m, 1H), 1.89-1.46 (m, 4H), 0.90 (s, 9H), 0.07 (s, 6H). ¹³C NMR (62.9 MHz, CDCl₃): δ 135.7, 128.8, 128.6, 127.8, 60.1, 55.1, 51.6, 47.5, 36.3, 28.1, 25.9, 18.1, −5.5. IR (neat, cm⁻¹): 1309, 1154, 1132, 1100, 1072, 947, 834, 782, 752, 704, 665. HRMS (ESI) ([M+H]⁺) Calcd. for C₁₈H₃₃N₂O₃SSiNa 407.1795. Found 407.1784.

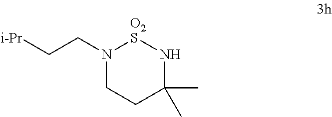

Purified by chromatography on silica gel (2:1 hexanes/EtOAc), white solid, TLC R_{f}=0.7 (1:1 hexanes/EtOAc). ¹H NMR (400 MHz, CDCl₃): δ 3.94 (s, 1H), 3.33-3.29 (m, 2H), 3.03 (t, J=7.2 Hz, 2H), 1.66-1.58 (m, 3H), 1.43 (q, J=7.2 Hz, 2H), 1.34 (s, 6H), 0.90 (d, J=6.8 Hz, 6H). ¹³C NMR (62.9 MHz, CDCl₃): δ 56.4, 46.6, 45.7, 36.2, 35.3, 28.5, 25.8, 22.4. IR (neat, cm⁻¹): 2924, 2854, 1323, 1161, 1143, 1086, 908, 754, 741, 627. HRMS (ESI) ([M+Na]⁺) Calcd. for C₁₀H₂₂N₂O₂SNa 257.1294. Found 257.1297.

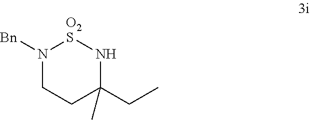

Purified by chromatography on silica gel (gradient elution: 4:1-2:1 hexanes/EtOAc), white solid, TLC R_{f}=0.20 (4:1 hexanes/EtOAc). ¹H NMR (250 MHz, CDCl₃): δ 7.35-7.29 (m, 5H), 4.25, 4.11 (AB q, J=13.8 Hz, each 1H), 4.17 (br, 1H), 3.26-3.10 (m, 2H), 1.88-1.73 (m, 1H), 1.60-1.38 (m, 3H), 1.30 (s, 3H), 0.93 (t, J=7.5 Hz, 3H). ¹³C NMR (62.9 MHz, CDCl₃): δ 135.5, 128.9, 128.7, 127.9, 59.4, 51.6, 44.8, 34.1, 33.8, 24.2, 7.6. IR (neat, cm⁻¹): 3234, 1425, 1338, 1323, 1297, 1169, 1143, 1088, 921, 861, 764, 730, 695. HRMS (ESI) ([M+Na]⁺) Calcd. for C₁₃H₂₀N₂O₂SNa 291.1138. Found 291.1129.

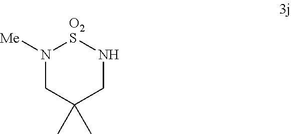

Purified by chromatography on silica gel (2:1 hexanes/EtOAc), white solid, TLC R_{f}=0.43 (1:1 hexanes/EtOAc). ¹H NMR (250 MHz, CDCl₃): δ 4.46-4.40 (m, 1H), 3.14 (d, J=7.8 Hz, 2H), 2.84 (s, 2H), 2.67 (s, 3H), 1.04 (s, 6H). ¹³C NMR (62.9 MHz, CDCl₃): δ 64.0, 55.8, 36.6, 31.2, 23.6. IR (neat, cm⁻¹): 3236, 2964, 1318, 1306, 1149, 1142, 1111, 1049, 910, 783, 739, 709. HRMS (ESI) ([M+Na]⁺) Calcd. for C₆H₁₄N₂O₂SNa 201.0068. Found 201.0672.

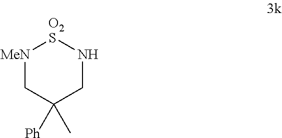

Purified by chromatography on silica gel (gradient elution: 4:1-2:1 hexanes/EtOAc), white solid, TLC R_{f}=0.63 (1:1 hexanes/EtOAc). ¹H NMR (400 MHz, CDCl₃): δ 7.38-7.24 (m, 5H), 4.23 (q, J=7.6 Hz, 1H), 3.76 (ddd, J=2.0, 6.4, 14.4 Hz, 1H), 3.56-3.47 (m, 2H), 3.16 (dd, J=0.8, 12.8 Hz, 1H), 2.75 (s, 3H), 1.35 (s, 3H). ¹³C NMR (100 MHz, CDCl₃): δ 142.3, 129.0, 127.3, 126.0, 62.0, 55.3, 38.5, 36.5, 24.3. IR (neat, cm⁻¹): 1316, 1147, 1099, 1045, 1030, 773, 761, 732, 699. HRMS (ESI) ([M+Na]⁺) Calcd. C₁₁H₁₆N₂O₂SNa For 263.0825 Found 263.0824.

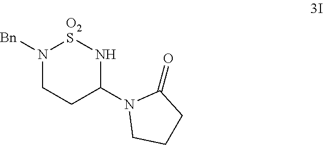

Purified by chromatography on silica gel (gradient elution: 30:1-20:1 EtOAc/MeOH), white solid, TLC R_{f}=0.45 (20:1 EtOAc/MeOH); ¹H NMR (250 MHz, CDCl₃): δ 7.33 (s, 5H), 5.70 (d, J=8.8 Hz, 1H), 5.11-5.00 (m, 1H), 4.39, 4.10 (AB q, J=13.8 Hz, each 1H), 3.58-3.42 (m, 2H), 3.28 (dt, J=2.8, 13.3 Hz, 1H), 3.15-3.06 (m, 1H), 2.43-2.23 (m, 3H), 2.12-1.99 (m, 2H), 1.68-1.58 (m, 1H). ¹³C NMR (62.9 MHz, CDCl₃): δ 176.5, 135.1, 128.7, 128.0, 66.4, 51.5, 46.3, 45.2, 31.5, 24.9, 18.4. IR (neat, cm⁻¹): 1663, 1427, 1352, 1168, 1093, 1074, 878, 781, 753. HRMS (ESI) ([M+Na]⁺) Calcd. for C₁₄H₂₀N₃O₃S 310.1220. Found 310.1222.

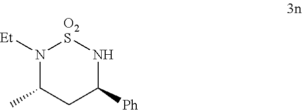

Purified by chromatography on silica gel (gradient elution: 4:1-2:1 hexanes/EtOAc), white solid, TLC R_{f}=0.20 (4:1 hexanes/EtOAc). ¹H NMR (400 MHz, CDCl₃): δ 7.37-7.29 (m, 5H), 4.94-4.87 (m, 1H), 4.14 (d, J=8.8 Hz, 1H), 3.79-3.71 (m, 1H), 3.44-3.34 (m, 1H), 3.21-3.11 (m, 1H), 2.15 (dt, J=5.6, 13.6 Hz, 1H), 1.86 (dt, J=2.4, 14.0 Hz, 1H), 1.50 (d, J=7.2 Hz, 3H), 1.20 (t, J=7.6 Hz, 3H). ¹³C NMR (100 MHz, CDCl₃): δ 139.4, 128.9, 128.3, 126.3, 54.9, 53.5, 42.5, 35.9, 16.8, 14.0. IR (neat, cm⁻¹): 2925, 1446, 1291, 1174, 1155, 1138, 1094, 1059, 950, 796, 738, 693. HRMS (ESI) ([M+H]⁺) Calcd. for C₁₂H₁₉N₂O₂S 255.1162. Found 255.1168.

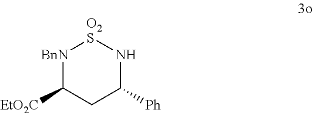

Purified by chromatography on silica gel (gradient elution: 4:1-2:1 hexanes/EtOAc), white solid, TLC R_{f}=0.30 (4:1 hexanes/EtOAc). ¹H NMR (250 MHz, CDCl₃): 7.46-7.30 (m, 10H), 5.12-5.02 (m, 1H), 4.70, 4.60 (AB q, J=14.0 Hz, each 1H), 4.32-4.17 (m, 3H), 3.90 (dd, J=2.0, 5.5 Hz, 1H), 2.41 (dq, J=14.0, 2.5, Hz, 1H), 1.90-1.76 (m, 1H), 1.35 (t, J=7.0 Hz, 3H). ¹³C NMR (62.9 MHz, CDCl₃): δ 170.1, 139.2, 135.1, 128.9, 128.8, 128.7, 128.5, 128.3, 126.2, 61.8, 57.0, 56.9, 52.3, 29.6, 14.1. IR (neat, cm⁻¹): 1722, 1418, 1345, 1327, 1215, 1157, 1025, 947, 805, 729, 695. HRMS (ESI) ([M+H]⁺) Calcd. for C₁₉H₂₃N₂O₄S 375.1373. Found 375.1388.

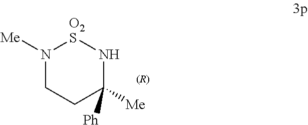

Purified by chromatography on silica gel (gradient elution: 2:1-1:1 hexanes/EtOAc), white solid, TLC R_{f}=0.25 (2:1 hexanes/EtOAc). ¹H NMR (400 MHz, CDCl₃): δ 7.47-7.18 (m, 5H), 4.26 (br, 1H), 3.53-3.42 (m, 1H), 3.32-3.26 (m, 1H), 2.75 (s, 3H), 2.41-2.33 (m, 1H), 2.09-2.02 (m, 1H), 1.64 (s, 3H). ¹³C NMR (62.9 MHz, CDCl₃): δ 145.0, 128.7, 127.7, 124.6, 61.8, 49.3, 36.3, 33.1, 29.7. IR (neat, cm⁻¹): 1329, 1312, 1165, 1133, 926, 754, 729, 697. HRMS (ESI) ([M+H]⁺) Calcd. for C₁₁H₁₇N₂O₂S 241.1005. Found 241.1010. HPLC analysis: ee=85%. (s, s) Whelk-O1 (80% hexanes: 20% isopropanol, 0.8 mL/min), tₘᵢₙₒᵣ=16.5 min, tₘₐⱼₒᵣ=13.8 min.

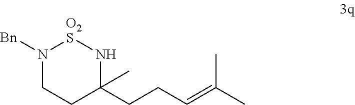

Purified by chromatography on silica gel (gradient elution: 10:1-4:1 hexanes/EtOAc), white solid, TLC R_{f}=0.27 (4:1 hexanes/EtOAc). ¹H NMR (250 MHz, CDCl₃): δ 7.35-7.27 (m, 5H), 5.11-5.03 (m, 1H), 4.25, 4.11 (AB q, J=13.5 Hz, each 1H), 4.01 (s, 1H), 3.26-3.09 (m, 2H), 2.22-1.91 (m, 2H), 1.83-1.38 (m, 4H), 1.67 (s, 3H), 1.60 (s, 3H), 1.35 (s, 3H). ¹³C NMR (62.9 MHz, CDCl₃): δ 135.5, 132.5, 128.8, 128.6, 127.9, 123.3, 59.1, 51.5, 44.6, 40.9, 34.5, 25.7, 24.9, 21.9, 17.7. IR (neat, cm⁻¹): 3246, 2968, 1456, 1321, 1161, 862, 768, 731, 697. HRMS (ESI) ([M+NH₄]⁺) Calcd. C₁₇H₃₀N₃O₂S For 345.1607 Found 345.1608. HPLC analysis: ee=90%. Chiralcel OD-H (95% hexanes: 5% isopropanol, 0.6 mL/min), tₘᵢₙₒᵣ=21.0 min, tₘₐⱼₒᵣ=23.9 min.

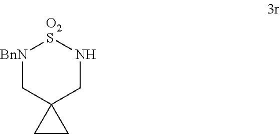

Purified by chromatography on silica gel (gradient elution: 30:1-20:1 DCM/EtOAc), colorless liquid, TLC R_{f}=0.67 (10:1 DCM/EtOAc). ¹H NMR (400 MHz, CDCl₃): δ 7.34-7.26 (m, 5H), 4.44 (t, J=7.2 Hz, 1H), 4.26 (s, 2H), 3.25 (d, J=7.2 Hz, 2H), 2.89 (s, 2H), 0.50 (dd, J=5.2, 5.6 Hz, 2H), 0.39 (dd, J=5.2, 5.6 Hz, 2H). ¹³C NMR (100 MHz, CDCl₃): δ 135.3, 128.6, 128.4, 127.9, 55.6, 52.9, 51.7, 16.2, 9.9. IR (neat, cm⁻¹): 1327, 1156, 1029, 788, 769, 730, 697. HRMS (ESI) ([M+H]⁺) Calcd. C₁₁H₁₇N₂O₂S For 253.1005 Found 253.1004.

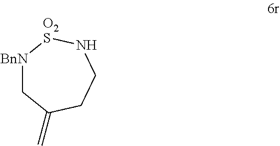

Purified by chromatography on silica gel (gradient elution: 30:1-20:1 DCM/EtOAc), TLC R_{f}=0.58 (10:1 DCM/EtOAc). ¹H NMR (400 MHz, CDCl₃): δ 7.37-7.27 (m, 5H), 5.01 (d, J=1.2 Hz, 1H), 4.81 (s, 1H), 4.54-4.49 (m, 1H), 4.38 (s, 2H), 3.72 (s, 2H), 3.25-3.20 (m, 2H), 2.53 (t, J=5.2 Hz, 2H). ¹³C NMR (100 MHz, CDCl₃): δ 141.8, 136.3, 128.7, 128.2, 127.8, 117.1, 51.2, 50.9, 41.9, 37.4. IR (neat, cm⁻¹): 1340, 1315, 1153, 925, 773, 738, 698. HRMS (ESI) ([M+Na]⁺) Calcd. C₁₁H₁₆N₂O₂SNa For 275.0825 Found 275.0826.

### Experimental Procedure and Characterization Data for Products 4m and 5m:

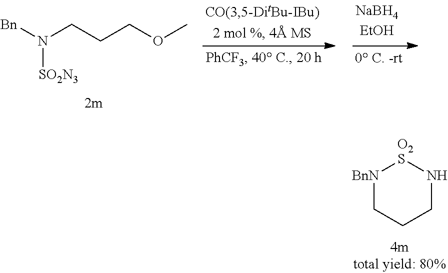

An oven dried Schlenk tube was charged with catalyst (0.002 mmol) and 4 Å MS (50 mg), then evacuated and back filled with nitrogen. The Teflon screw cap was replaced with a rubber septum and then an approximately 0.2 ml portion of PhCF₃ was added, then azide (0.1 mmol), followed by the remaining PhCF₃ (total 0.5 mL). The Schlenk tube was then purged with nitrogen for 2 minutes and the rubber septum was replaced with a Teflon screw cap. The Schlenk tube was then placed in an oil bath at 40° C. for 20 h. After completion of the reaction, EtOH (0.5 ml) was added to the reaction mixture, then NaBH₄ (4 eq, 0.4 mmol) was slowly added to the reaction mixture at 0° C. The reaction was allowed to come to room temperature and was stirred at room temperature for 4 h. The reaction mixture was quenched upon addition of 1 mL of H₂O. The reaction mixture was transferred to a separatory funnel, and the organic phase was collected. The aqueous layer was extracted with 3×3 mL of CH₂Cl₂. The combined organic extracts dried over Na₂SO₄, and concentrated under reduced pressure. Purified by chromatography on silica gel (gradient elution: 2:1-1:1 hexanes/EtOAc), white solid, TLC R_{f}=0.33 (2:1 hexanes/EtOAc). ¹H NMR (250 MHz, CDCl₃): δ 7.34-7.30 (m, 5H), 4.20 (s, 3H), 3.51 (q, J=7.0 Hz, 2H), 3.18 (t, J=5.5 Hz, 2H), 1.72-1.59 (m, 2H). ¹³C NMR (62.9 MHz, CDCl₃): δ 135.4, 128.8, 128.6, 127.9, 51.9, 48.4, 45.3, 23.8. IR (neat, cm⁻¹): 1493, 1407, 1331, 1305, 1164, 1134, 1094, 860, 803, 751, 616. HRMS (ESI) ([M+H]⁺) Calcd. for C₁₀H₁₅N₂O₂S 227.0849. Found 227.0856.

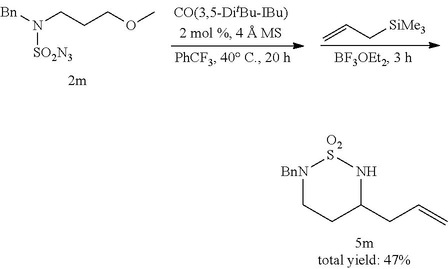

An oven dried Schlenk tube was charged with catalyst (0.002 mmol), and 4 Å MS (50 mg), then evacuated and back filled with nitrogen. The Teflon screw cap was replaced with a rubber septum and then an approximately 0.2 ml portion of PhCF₃ was added, then azide (0.1 mmol), followed by the remaining PhCF₃ (total 0.5 mL). The Schlenk tube was then purged with nitrogen for 2 minutes and the rubber septum was replaced with a Teflon screw cap. The Schlenk tube was then placed in an oil bath at 40° C. for 20 h. After completion of the reaction, allyltrimethylsilane (0.4 mmol, 4.0 equiv) in DCM (0.5 ml) was added to the reaction mixture. To this mixture, 100 μL solution of BF₃.OEt₂ (0.2 mmol, 2 equiv) in CH₂Cl₂ was added dropwise over 30 min. The reaction was stirred at room temperature for 4 h. The reaction mixture was diluted with 5 mL of CH₂Cl₂, quenched upon addition of 1 mL of saturated aqueous NaHCO₃, transferred to a separatory funnel, and the organic phase was collected. The aqueous layer was extracted with 2×3 mL of CH₂Cl₂. The combined organic extracts were dried over Na₂SO₄ and concentrated under reduced pressure. The crude product was purified by chromatography on silica gel (gradient elution: 4:1-2:1 hexanes/EtOAc), white solid, TLC R_{f}=0.32 (4:1 hexanes/EtOAc). ¹H NMR (400 MHz, CDCl₃): δ 7.36-7.26 (m, 5H), 5.79-5.67 (m, 1H), 5.18-5.12 (m, 2H), 4.47, 3.92 (AB q, J=13.6 Hz, each 1H), 3.89 (br, 1H), 3.75-3.67 (m, 1H), 3.28 (dt, J=3.2, 12.8 Hz, 1H), 3.11-3.05 (m, 1H), 2.28 (t, J=6.8 Hz, 2H), 1.63-1.50 (m, 2H). ¹³C NMR (62.9 MHz, CDCl₃): δ 135.4, 132.2, 128.8, 128.6, 127.9, 119.7, 55.5, 51.7, 47.5, 39.4, 28.5. IR (neat, cm⁻¹): 1420, 1330, 1162, 1098, 920, 857, 768, 728, 697. HRMS (ESI) ([M+Na]⁺) Calcd. for C₁₃H₁₈N₂O₂SNa 289.0981. Found 289.0985.

### General Procedure for Deprotection of Sulfone Group:

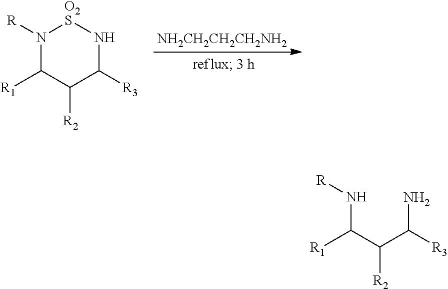

An oven dried Schlenk tube was charged with 0.1 mmol of amine in 0.3 mL of dry 1,3-diaminopropane. The Teflon screw cap was replaced with a rubber septum. The Schlenk tube was then purged with nitrogen for 2 minutes and the rubber septum was replaced with a Teflon screw cap. The mixture was heated on an oil bath at 145° C. and refluxed for 3 h. After cooling to room temperature, the mixture was diluted with 5 mL of CH₂Cl₂, and extracted with 5 mL of water. After extraction of the water layer with 4×5 mL of CH₂Cl₂, the combined organic layers were dried over Na₂SO₄. The product was obtained as a colorless oil in high yield after removal of the solvent under reduced pressure.

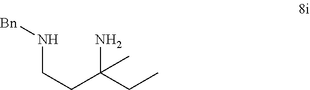

¹H NMR (250 MHz, CDCl₃): δ 7.27-7.17 (m, 5H), 3.73 (s, 2H), 2.68-2.61 (m, 2H), 1.53-1.46 (m, 5H), 1.36-1.18 (m, 2H), 0.96 (s, 3H), 0.80 (t, J=7.5 Hz, 3H). ¹³C NMR (62.9 MHz, CDCl₃): δ 140.3, 128.4, 128.1, 126.9, 54.3, 51.3, 45.3, 41.6, 35.8, 27.6, 8.3. IR (neat, cm⁻¹): 2195, 1540, 1521, 1508, 1473, 1403, 1316, 1282, 693. HRMS (ESI) ([M+H]⁺) Calcd. For C₁₃H₂₃N₂ 207.1856 Found 207.1853.

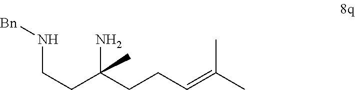

¹H NMR (250 MHz, CDCl₃): δ 7.31-7.20 (m, 5H), 5.11-5.03 (m, 1H), 3.77 (s, 2H), 2.70 (t, J=7.5 Hz, 2H), 2.01-1.90 (m, 2H), 1.66 (d, J=0.8 Hz, 3H), 1.60-1.53 (m, 2H), 1.57 (s, 3H), 1.37-1.23 (m, 5H), 1.04 (s, 3H). ¹³C NMR (62.9 MHz, CDCl₃): δ 140.4, 131.4, 128.4, 128.1, 126.9, 124.4, 54.3, 51.3, 45.3, 43.5, 42.2, 28.2, 25.7, 22.7, 17.6. IR (neat, cm⁻¹): 2968, 1453, 1375, 1261, 1144, 1106, 1028, 972, 736, 699. HRMS (ESI) ([M+H]⁺) Calcd. For C₁₇H₂₉N₂ 261.2325. Found 261.2317.

### REFERENCES

(1)(a) Chiral Amine Synthesis: Methods, Developments and Applications; Nugent, T. C., Ed., Willey-VCH: Weinheim; 2010. (b) Amino Group Chemistry: From Synthesis to the Life Sciences; Racci, A., Ed., Willey-VCH: Weinheim; 2008. (c) Modern Amination Methods; Racci, A., Ed., Willey-VCH: Weinheim; 2000. (d) Salvatore, R. N.; Yoon, C. H.; Jung, K. W. Tetrahedron 2001, 57, 7785. (e) Johannsen, M.; Jorgensen, K. A. Chem. Rev. 1998, 98, 1689. (f) Muller, T. E.; Beller, M. Chem. Rev. 1998, 98, 675.
(2)(a) Collet, F.; Dodd, R. H.; Dauban, P. Chem. Commun. 2009, 5061. (b) Davies, H. M. L.; Manning, J. R. Nature 2008. 451, 417. (c) Davies, H. M. L. Angew. Chem. Int. Ed. 2006, 45, 6422. (d) Espino, C. G.; Du Bois, J. In Modern Rhodium-Catalyzed Organic Reactions; Evans, P. A., Ed.; Wiley-VCH; Weinheim, 2005; pp 379-416. (e) Davies, H. M. L.; Long, M. S. Angew. Chem. Int. Ed. 2005, 44, 3518. (f) Halfen, J. A. Curr. Org. Chem. 2005, 9, 657. (g) Muller, P.; Fruit, C. Chem. Rev. 2003, 103, 2905.
(3) For early examples of in situ iminoiodane generation for catalytic nitrene transfers, see: (a) Yu, X.-Q.; Huang, J.-S.; Zhou, X.-G.; Che, C.-M. Org. Lett. 2000, 2, 2233. (b) Dauban, P.; Saniere, L.; Tarrade, A.; Dodd, R. H. J. Am. Chem. Soc. 2001, 123, 7707. (c) Espino, C. G.; Du Bois, J. Angew. Chem. Int. Ed. 2001, 40, 598.
(4) Kurokawa, T.; Kim, M.; Du Bois, J. Angew. Chem. Int. Ed. 2009, 48, 2777.
(5) For the only example of Rh2-catalyzed intramolecular amination of a primary C—H bond: N-tosyloxycarbamate provided a yield of 41%, see: Huard, K.; Lebel, H. Chem. Eur. J. 2008, 14, 6222.
(6)(a) Ruppel, J. V.; Kamble, R. M.; Zhang, X. P. Org. Lett. 2007, 9, 4889. (b) Lu, H.; Tao, J.; Jones, J. E.; Wojtas, L.; Zhang, X. P. Org. Lett. 2010, 12 1248. (c) Lu, H.; Subbarayan, V.; Tao, J.; Zhang, X. P. Organometallics 2010, 29, 389.
(7) Azides, a broad class of well-known compounds that are widely available from straightforward synthesis,⁸ could potentially serve as alternative nitrene sources for metal-catalyzed nitrene transfer reactions, including C—H amination.⁹ In addition to generating chemically stable and environmentally benign nitrogen gas as the only byproduct, azide-based nitrene-transfers could operate under neutral and nonoxidative conditions. Despite these potential advantages, catalytic C—H amination with azides is largely underdeveloped as they are generally considered to be ineffective toward metal-mediated decomposition.¹⁰
(8)(a) Scriven, E. F. V.; Turnbull, K. Chem. Rev. 1988, 88, 297. (b) Brase, S.; Gil, C.; Knepper, K.; Zimmermann, V. Angew. Chem. Int. Ed. 2005, 44, 5188.
(9)(a) Katsuki, T. Chem. Lett. 2005, 1304. (b) Cenini, S.; Gallo, E.; Caselli, A.; Ragaini, F.; Fantauzzi, S.; Piangiolino, C. Coord. Chem. Rev. 2006, 250, 1234.
(10)(a) Reddy, R. P.; Davies, H. M. L. Org. Lett. 2006, 8, 5013. (b) Lebel, H.; Leogane, O.; Huard, K.; Lectard, S. Pure Appl. Chem. 2006, 78, 363.
(11) For examples of Rh2-catalyzed intramolecular C—H amination of vinyl and aryl azides, see: (a) Stokes, B. J.; Dong, H.; Leslie, B. E.; Pumphrey, A. L.; Driver, T. G. J. Am. Chem. Soc. 2007, 129, 7500. (b) Shen, M.; Leslie, B. E.; Driver, T. G. Angew. Chem. Int. Ed. 2008, 47, 5056.
(12)(a) Matier, W. L.; Corner, W. T.; Deitchman, D. J. Med. Chem. 1972, 15, 538. (b) Griffiths, J. J. Chem. Soc. (C), 1971, 3191. (c) Goddard-Borger, E. D.; Stick, R. V. Org. Lett. 2007, 9, 3797.
(13) Sulfamoyl azides were reported to be chemically stable, even in strong acidic and basic conditions.12 Our DSC experiments indicated that these sulfamoyl azides were thermally stable without decomposition up to at least 100° C.; see Supporting Information for a representative DSC plot.
(14)(a) Ruppel, J. V.; Jones, J. E.; Huff, C. A.; Kamble, R. M.; Chen, Y.; Zhang, X. P. Org. Lett. 2008, 10, 1995. (b) Subbarayan, V.; Ruppel, J. V.; Zhu, S.-F.; Perman, J. A.; Zhang, X. P. Chem. Commun. 2009, 4266.
(15) Fiori, K. W.; Fleming, J. J.; Du Bois, J. Angew. Chem. Int. Ed. 2004, 43, 4349.
(16) Luo, Y. R. Handbook of Bond Dissociation Energies in Organic Compounds; CRC Press: Boca Raton, Fla., 2003.
(17)(a) Reitz, A. B.; Smith, G. R.; Parker, M. H. Expert Opin. Ther. Patents 2009, 19, 1449. (b) Winum, J.-Y.; Scozzafava, A.; Montero, J.-L.; Supuran, C. T. Expert Opin. Ther. Patents 2006, 16, 27.
(18) Jagt, R. B. C.; Toullec, P. Y.; Geerdink, D.; de Vries, J. G.; Fering a, B. L.; Minnaard, A. J. Angew. Chem. Int. Ed. 2006, 45, 2789.

What is claimed is:

1. A sulfamoyl azide corresponding to Formula SA-1: 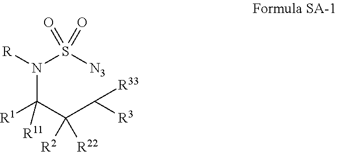
wherein
R is hydrogen, hydrocarbyl, substituted hydrocarbyl, heterocyclo, or, in combination with R¹ or R¹¹ and carbon atom to which they are attached, form a heterocyclo,
R¹ is hydrogen, hydrocarbyl, substituted hydrocarbyl, heterocyclo, or in combination with R and carbon atoms to which R and R¹, respectively, are attached, form a heterocyclo,
R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,
R³ is hydrogen, hydrocarbyl, substituted hydrocarbyl, or heterocyclo,
R¹¹ is hydrogen, hydrocarbyl, substituted hydrocarbyl, heterocyclo, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

2. The sulfamoyl azide of claim 1, the sulfamoyl azide corresponding to formula SA-2 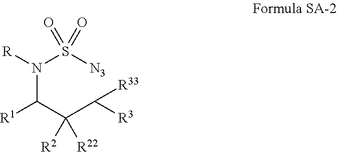
wherein
R is hydrogen, hydrocarbyl, substituted hydrocarbyl, heterocyclo, or, in combination with R¹ and carbon atom to which they are attached, form a heterocyclo,
R¹ is hydrogen, hydrocarbyl, substituted hydrocarbyl, heterocyclo, or in combination with R and carbon atoms to which R and R¹, respectively, are attached, form a heterocyclo,
R² is hydrogen, hydrocarbyl, substituted hydrocarbyl, or heterocyclo,
R³ is hydrogen, hydrocarbyl, substituted hydrocarbyl, or heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

3. The sulfamoyl azide of claim 1, the sulfamoyl azide corresponding to formula SA-2-2 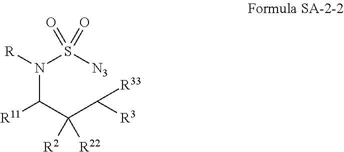
wherein
R is hydrogen, hydrocarbyl, substituted hydrocarbyl, or heterocyclo,
R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,
R³ is hydrogen, hydrocarbyl, substituted hydrocarbyl, or heterocyclo,
R¹¹ is hydrogen, hydrocarbyl, substituted hydrocarbyl, heterocyclo, or in combination with R² and carbon atoms to which R² and R¹¹, respectively, are attached, form a heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

4. The sulfamoyl azide of claim 1, the sulfamoyl azide corresponding to formula SA-3 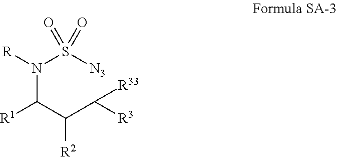
wherein
R is hydrogen, hydrocarbyl, substituted hydrocarbyl, or heterocyclo,
R¹ is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R³³ is hydrogen, hydrocarbyl, substituted hydrocarbyl, or heterocyclo.

5. The sulfamoyl azide of claim 1, the sulfamoyl azide corresponding to formula SA-4 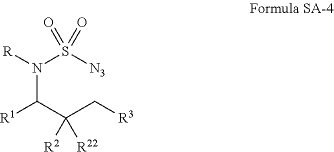
wherein
R is hydrogen, hydrocarbyl, substituted hydrocarbyl, or heterocyclo,
R¹ is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R²² is hydrogen, hydrocarbyl, substituted hydrocarbyl, or heterocyclo.

6. The sulfamoyl azide of claim 1, the sulfamoyl azide corresponding to formula SA-5 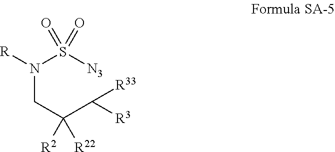
wherein
R is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo,
R²² is hydrogen, hydrocarbyl, substituted hydrocarbyl, or heterocyclo, and
R³³ is hydrogen, hydrocarbyl, substituted hydrocarbyl, or heterocyclo.

7. The sulfamoyl azide of claim 1, the sulfamoyl azide corresponding to formula SA-6 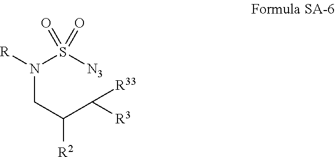
wherein
R is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R³³ is hydrogen, hydrocarbyl, substituted hydrocarbyl, or heterocyclo.

8. The sulfamoyl azide of claim 1, the sulfamoyl azide corresponding to formula SA-7 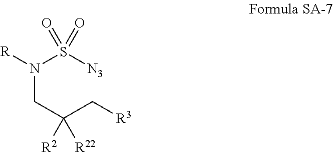
wherein
R is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R²² is hydrogen, hydrocarbyl, substituted hydrocarbyl, or heterocyclo.

9. The sulfamoyl azide of claim 1, the sulfamoyl azide corresponding to formula SA-9 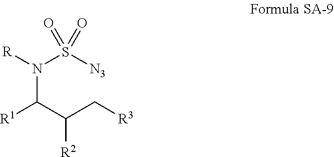
wherein
R is hydrogen, hydrocarbyl, substituted hydrocarbyl, or heterocyclo,
R¹ is hydrogen, hydrocarbyl, substituted hydrocarbyl, or heterocyclo,
R² is hydrogen, hydrocarbyl, substituted hydrocarbyl, or heterocyclo, and
R³ is hydrogen, hydrocarbyl, substituted hydrocarbyl, or heterocyclo.

10. A cyclic sulfonamide corresponding to Formula S-1: 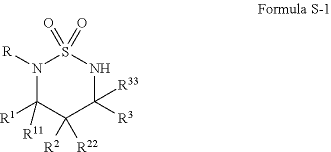
wherein
R is hydrogen, hydrocarbyl, substituted hydrocarbyl, heterocyclo, or, in combination with R¹ or R¹¹ and carbon atom to which they are attached, form a heterocyclo,
R¹ is hydrogen, hydrocarbyl, substituted hydrocarbyl, heterocyclo, or in combination with R and carbon atoms to which R and R¹, respectively, are attached, form a heterocyclo,
R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,
R³ is hydrogen, hydrocarbyl, substituted hydrocarbyl, or heterocyclo,
R¹¹ is hydrogen, hydrocarbyl, substituted hydrocarbyl, heterocyclo, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

11. The cyclic sulfonamide of claim 10, the cyclic sulfonamide corresponding to formula S-2 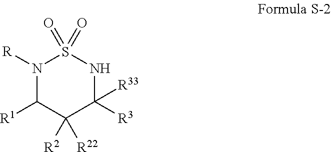
wherein
R is hydrogen, hydrocarbyl, substituted hydrocarbyl, heterocyclo, or, in combination with R¹ and carbon atom to which they are attached, form a heterocyclo,
R¹ is hydrogen, hydrocarbyl, substituted hydrocarbyl, heterocyclo, or in combination with R and carbon atoms to which R and R¹, respectively, are attached, form a heterocyclo,
R² is hydrogen, hydrocarbyl, substituted hydrocarbyl, or heterocyclo,
R³ is hydrogen, hydrocarbyl, substituted hydrocarbyl, or heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

12. The cyclic sulfonamide of claim 10, the cyclic sulfonamide corresponding to formula S-2-2 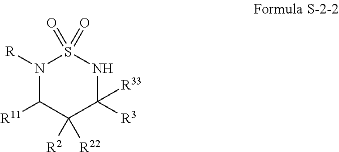
wherein
R is hydrogen, hydrocarbyl, substituted hydrocarbyl, or heterocyclo,
R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,
R³ is hydrogen, hydrocarbyl, substituted hydrocarbyl, or heterocyclo,
R¹¹ is hydrogen, hydrocarbyl, substituted hydrocarbyl, heterocyclo, or in combination with R² and carbon atoms to which R² and R¹¹, respectively, are attached, form a heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

13. The cyclic sulfonamide of claim 10, the cyclic sulfonamide corresponding to formula S-3 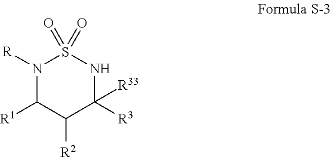
wherein
R is hydrogen, hydrocarbyl, substituted hydrocarbyl, or heterocyclo,
R¹ is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R³³ is hydrogen, hydrocarbyl, substituted hydrocarbyl, or heterocyclo.

14. The cyclic sulfonamide of claim 10, the cyclic sulfonamide corresponding to formula S-4 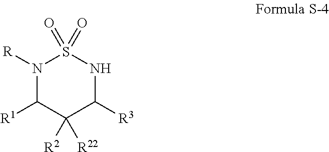
wherein
R is hydrogen, hydrocarbyl, substituted hydrocarbyl, or heterocyclo,
R¹ is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R²² is hydrogen, hydrocarbyl, substituted hydrocarbyl, or heterocyclo.

15. The cyclic sulfonamide of claim 10, the cyclic sulfonamide corresponding to formula S-5 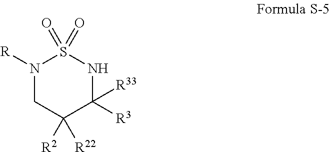
wherein
R is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo,
R²² is hydrogen, hydrocarbyl, substituted hydrocarbyl, or heterocyclo, and
R³³ is hydrogen, hydrocarbyl, substituted hydrocarbyl, or heterocyclo.

16. The cyclic sulfonamide of claim 10, the cyclic sulfonamide corresponding to formula S-6 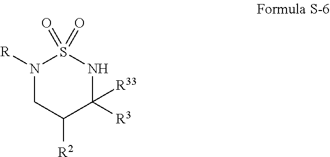
wherein
R is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R³³ is hydrogen, hydrocarbyl, substituted hydrocarbyl, or heterocyclo.

17. The cyclic sulfonamide of claim 10, the cyclic sulfonamide corresponding to formula S-7 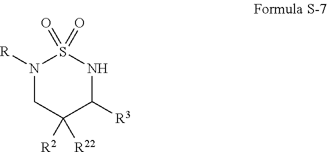
wherein
R is hydrogen, hydrocarbyl, substituted hydrocarbyl, or heterocyclo,
R² is hydrogen, hydrocarbyl, substituted hydrocarbyl, or heterocyclo,
R³ is hydrogen, hydrocarbyl, substituted hydrocarbyl, or heterocyclo, and
R²² is hydrogen, hydrocarbyl, substituted hydrocarbyl, or heterocyclo.

18. The cyclic sulfonamide of claim 10, the cyclic sulfonamide corresponding to formula S-9 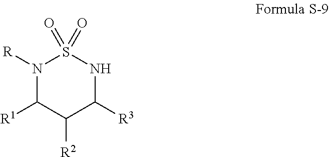
wherein
R is hydrogen, hydrocarbyl, substituted hydrocarbyl, or heterocyclo,
R¹ is hydrogen, hydrocarbyl, substituted hydrocarbyl, or heterocyclo,
R² is hydrogen, hydrocarbyl, substituted hydrocarbyl, or heterocyclo, and
R³ is hydrogen, hydrocarbyl, substituted hydrocarbyl, or heterocyclo.

19. A process for the preparation of a cyclic sulfonamide comprising the intramolecular C—H amination of a sulfamoyl azide in the presence of a cobalt porphyrin catalyst.

20. The process of claim 18 wherein the sulfamoyl azide corresponds to Formula SA-1 and the cyclic sulfonamide corresponds to Formula S-1: 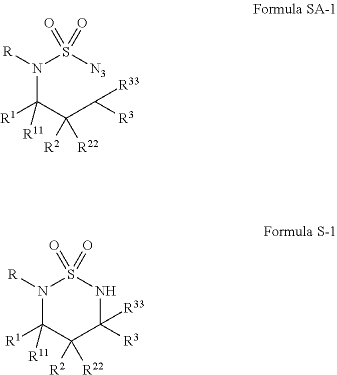
wherein
R is hydrogen, hydrocarbyl, substituted hydrocarbyl, heterocyclo, or, in combination with R¹ or R¹¹ and carbon atom to which they are attached, form a heterocyclo,
R¹ is hydrogen, hydrocarbyl, substituted hydrocarbyl, heterocyclo, or in combination with R and carbon atoms to which R and R¹, respectively, are attached, form a heterocyclo,
R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,
R³ is hydrogen, hydrocarbyl, substituted hydrocarbyl, or heterocyclo,
R¹¹ is hydrogen, hydrocarbyl, substituted hydrocarbyl, heterocyclo, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.

21. The process of claim 20 wherein the cobalt porphyrin catalyst is a D₂ₕ-symmetric porphyrin.

22. The process of claim 20 wherein the cobalt porphyrin catalyst is selected from the group consisting of

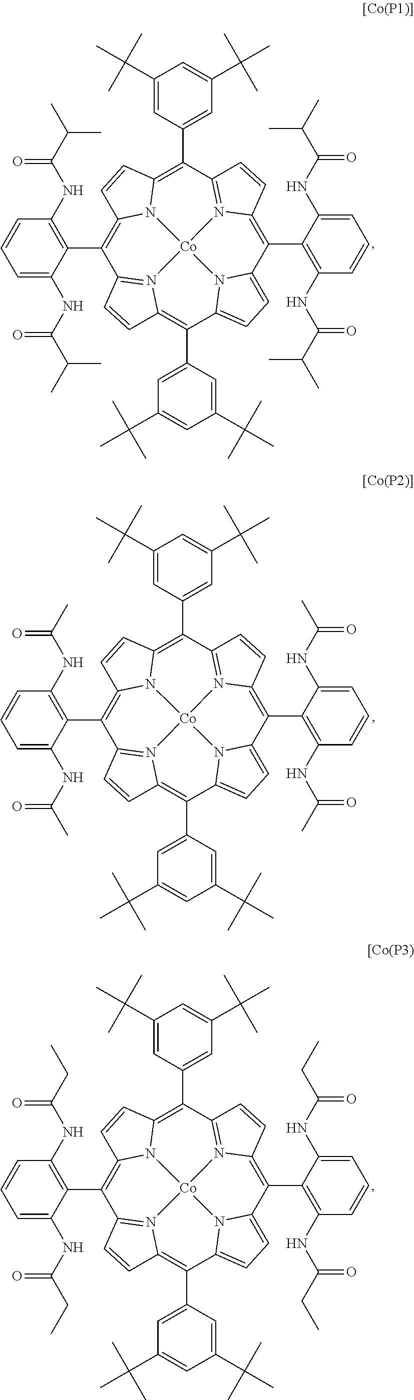

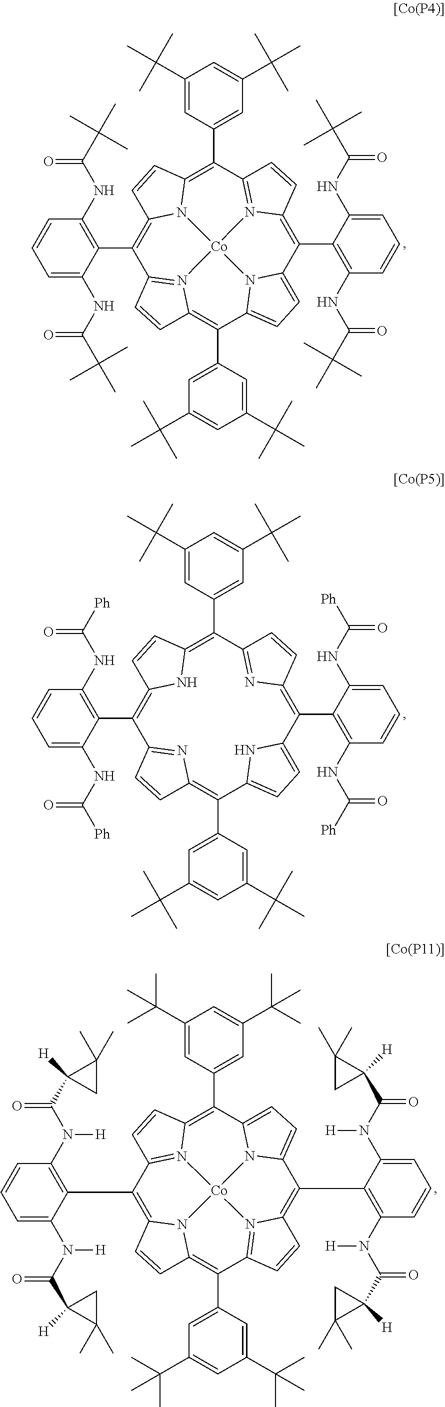

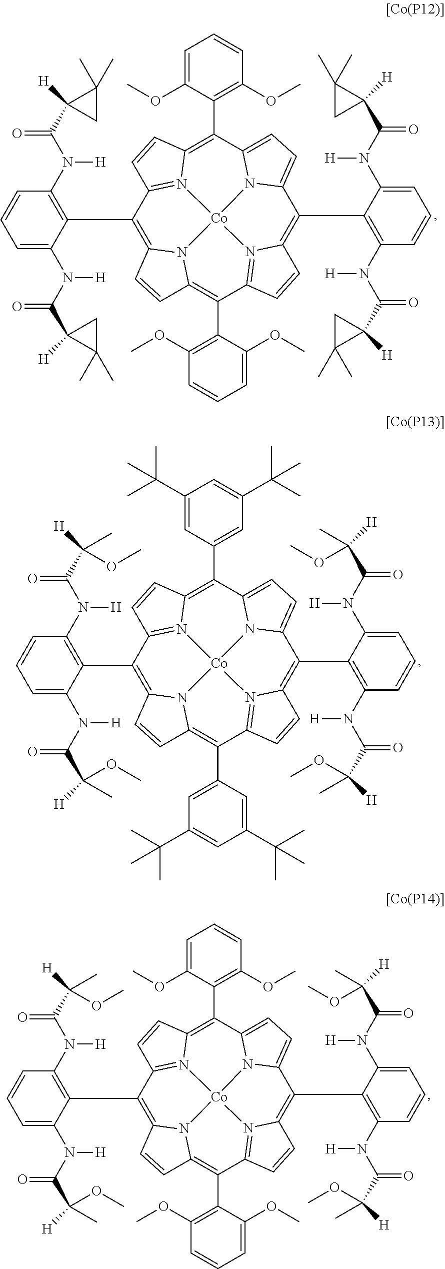

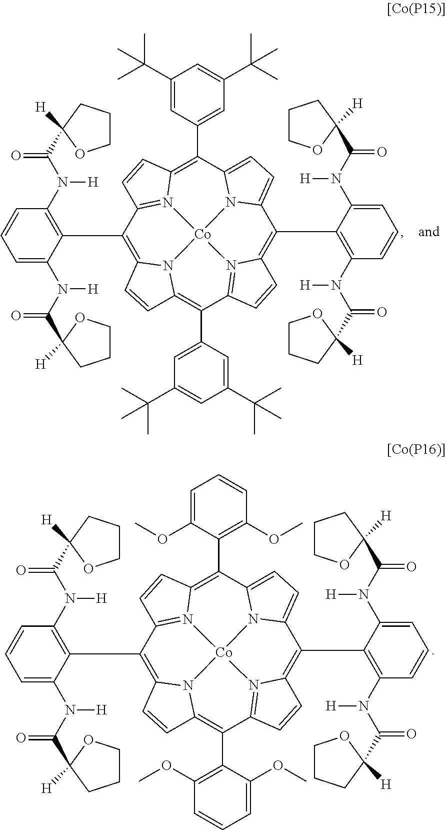

23. The process of claim 19 wherein the cobalt porphyrin catalyst is a D₂ₕ-symmetric porphyrin.

24. The process of claim 19 wherein the cobalt porphyrin catalyst is selected from the group consisting of

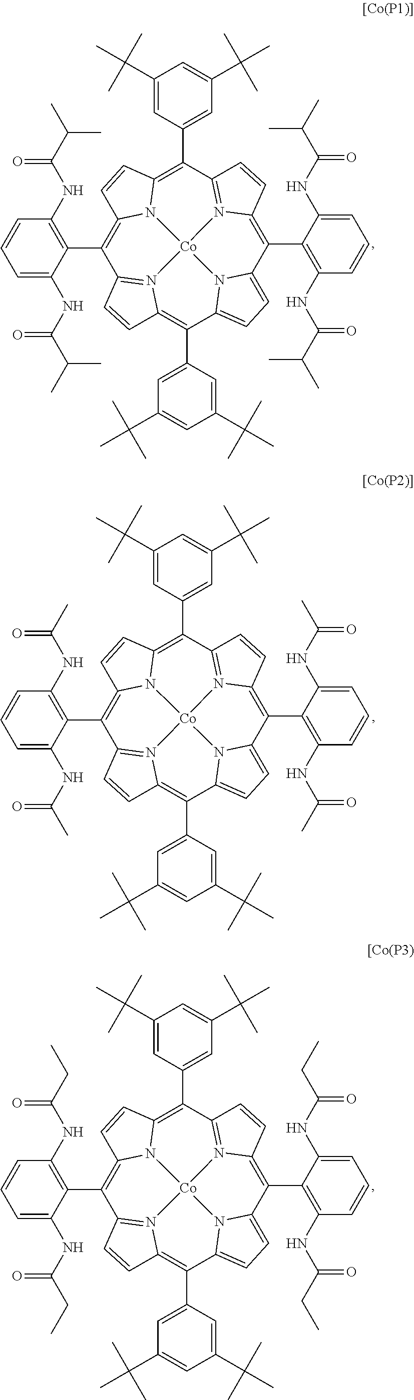

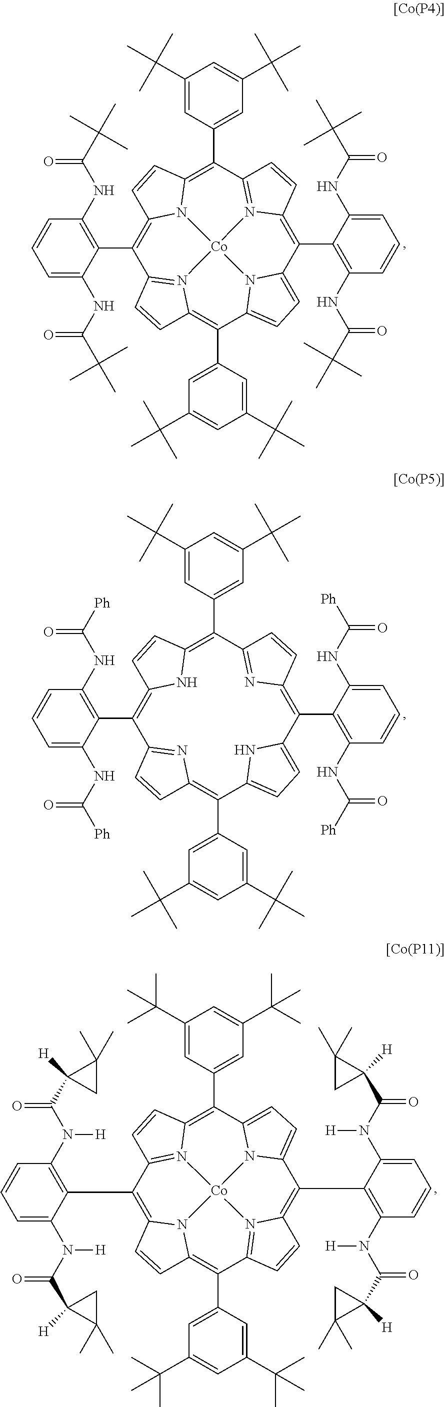

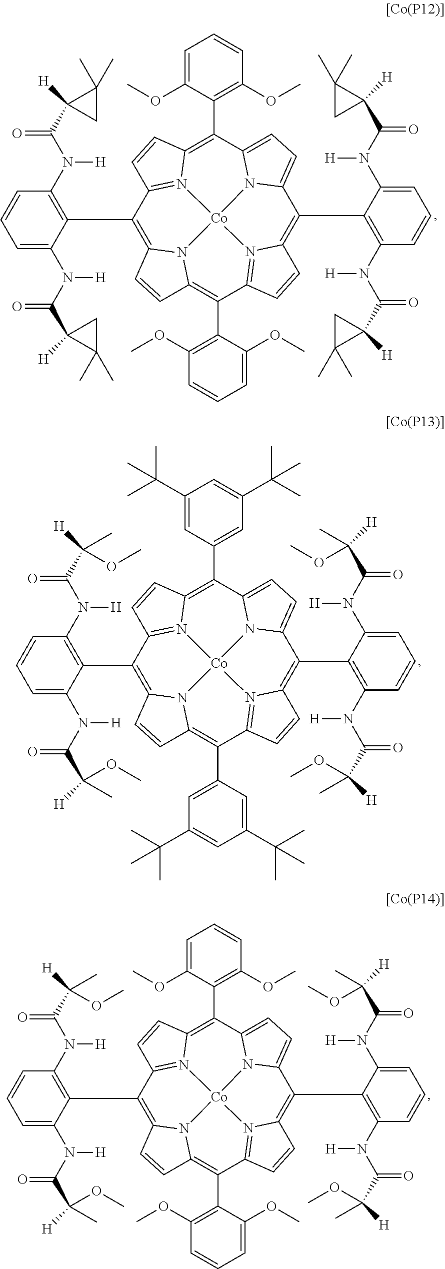

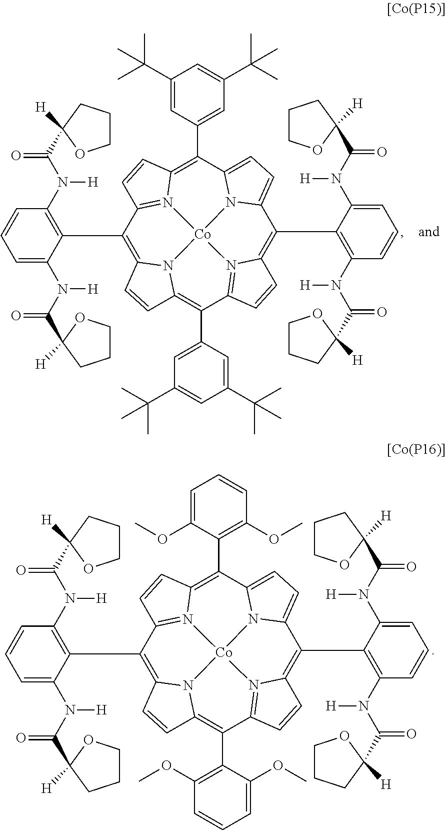

25. A process for the preparation of a diamine, the process comprising the transamination of a cyclic sulfonamide.

26. The process of claim 25 wherein the cyclic sulfonamide corresponds to Formula S-1 and the diamine corresponds to Formula A-1: 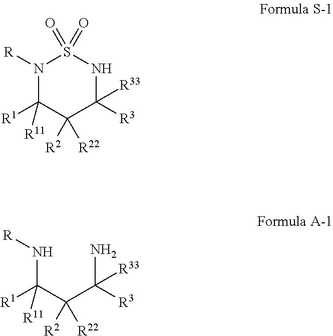
wherein
R is hydrogen, hydrocarbyl, substituted hydrocarbyl, heterocyclo, or, in combination with R¹ or R¹¹ and carbon atom to which they are attached, form a heterocyclo,
R¹ is hydrogen, hydrocarbyl, substituted hydrocarbyl, heterocyclo, or in combination with R and carbon atoms to which R and R¹, respectively, are attached, form a heterocyclo,
R² is hydrogen, hydrocarbyl, substituted hydrocarbyl, heterocyclo, or in combination with R¹¹ and the carbon atoms to which R² and R¹¹, respectively, are attached, form a carbocyclo or a heterocyclo,
R³ is hydrogen, hydrocarbyl, substituted hydrocarbyl, or heterocyclo,
R¹¹ is hydrogen, hydrocarbyl, substituted hydrocarbyl, heterocyclo, in combination with R and the carbon atoms to which R¹¹ and R, respectively, are attached, form a carbocyclo or a heterocyclo, or in combination with R² and the carbon atoms to which R¹¹ and R², respectively, are attached, form a carbocyclo or a heterocyclo,
R²² is hydrogen, hydrocarbyl, substituted hydrocarbyl, heterocyclo, or in combination with R³³ and the carbon atoms to which R²² and R³³, respectively, are attached, form a carbocyclo or a heterocyclo, and
R³³ is hydrogen, hydrocarbyl, substituted hydrocarbyl, heterocyclo, or in combination with R²² and the carbon atoms to which R²² and R³³, respectively, are attached, form a carbocyclo or a heterocyclo.
